(19) United States
(12) Patent Application Publication
(10) Pub. No.: US 2025/0026746 A1
(43) Pub. Date: Jan. 23, 2025

(54) ENHANCERS OF PARTICULATE GUANYLYL CYCLASE RECEPTOR A

(71) Applicants: Mayo Foundation for Medical Education and Research, Rochester, MN (US); University of Florida Research Foundation, Inc., Orlando, FL (US)

(72) Inventors: John C. Burnett, Jr., St. Anthony, MN (US); Sasantha J. Sangaralingham, Rochester, MN (US); Siobhan Malany, Orlando, FL (US); Satyamaheshwar Peddibhotla, Gainesville, FL (US)

(21) Appl. No.: 18/700,468

(22) PCT Filed: Nov. 22, 2022
(86) PCT No.: PCT/US2022/050674

§ 371 Date: Apr. 11, 2024

Related U.S. Application Data
(60) Provisional application No. 63/282,536, filed on Nov. 23, 2021.

(51) Int. Cl. C07D 417/12; A61K 31/454; A61P 9/12

(52) U.S. Cl. CPC C07D 417/12; A61K 31/454; A61P 9/12

(57) ABSTRACT

In some embodiments, the present disclosure provides a compound(S) or (R)-N-(4.6-difluorobenzo[d]thiazol-2-yl)-1-(2-(dimethylamino)ethyl) piperidine-3-carboxamide, as described herein, or a pharmaceutically acceptable salt thereof. Pharmaceutical compositions comprising the compound, and methods of treating. e.g., metabolic diseases using the compound are also provided.

### CLAIM OF PRIORITY

This application claims priority to U.S. Provisional Patent Application Ser. No. 63/282,536, filed on Nov. 23, 2021, the entire contents of which are hereby incorporated by reference.

### FEDERALLY SPONSORED RESEARCH AND DEVELOPMENT

This invention was made with government support under Grant nos. HL132854, HL136340, HL158548, and AG056315 awarded by the National Institutes of Health. The government has certain rights in the invention.

### TECHNICAL FIELD

This invention relates to organic compounds, and more particularly to 4-halobenzo[d]thiazole compounds useful in treating various conditions such as cardiovascular, renal, and metabolic diseases, as well as cancer.

### BACKGROUND

Metabolic disease continues to grow worldwide, representing one of the greatest burdens in human health. Metabolic disease, often referred to as metabolic syndrome, encompasses obesity, type 2 diabetes (T2DM), insulin resistance, hyperlipidemia and hypertension, and represents a global challenge to human health.

Cardiovascular disease (CVD), including myocardial infarction, stroke, and hypertension, also presents a significant socioeconomic burden. CVD remains the leading cause of death in the United States. The rates of CVD mortality per 100,000 people are currently nearly 400 for women, and nearly 700 for men.

Likewise, renal (kidney) disease is associated with a tremendous economic burden. High-income countries typically spend more than 2-3% of their annual health-care budget on the treatment of end-stage kidney disease, even though those receiving such treatment represent under 0.03% of the total population.

Finally, cancer is one of the leading causes of death in contemporary society. The numbers of new cancer cases and deaths is increasing each year. Currently, cancer incidence is nearly 450 cases of cancer per 100,000 men and women per year, while cancer mortality is nearly 71 cancer deaths per 100,000 men and women per year.

### SUMMARY

Atrial (ANP) and B-type natriuretic peptide (BNP) bind to the particulate guanylyl cyclase receptor A (pGC-A) that is highly expressed in heart, kidney, adrenals, vasculature and adipocytes. Following pGC-A activation, the second messenger 3′, 5′ cyclic guanosine monophosphate (cGMP) is produced resulting in widespread actions, including blood pressure lowering, renal enhancing, cardioprotective, and renin-angiotensin-aldosterone system (RAAS) suppressing properties. Advantageous metabolic actions of pGC-A include lipolysis, browning of adipocytes, stimulation of skeletal muscle energetics and release of adipokines such as adiponectin. The present disclosure is based, at least in part, on the realization that 4-halobenzo[d]thiazole compounds are positive allosteric modulators of pGC-A, and, therefore, are useful in treating cardiovascular, renal, and metabolic diseases. As a further advantage, the compounds of the present disclosure are orally bioavailable.

In one general aspect, the present disclosure provides a compound(S)-N-(4,6-difluorobenzo[d]thiazol-2-yl)-1-(2-(dimethylamino)ethyl) piperidine-3-carboxamide having formula:

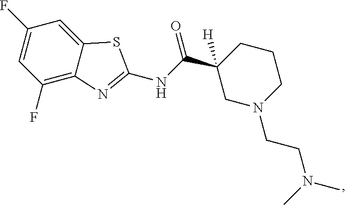 [SMILES: CN(C)CCN1CCC[C@H](C(=O)Nc2nc3c(F)cc(F)cc3s2)C1]

or a pharmaceutically acceptable salt thereof.

In another general aspect, the present disclosure provides a compound (R)-N-(4,6-difluorobenzo[d]thiazol-2-yl)-1-(2-(dimethylamino)ethyl) piperidine-3-carboxamide having formula:

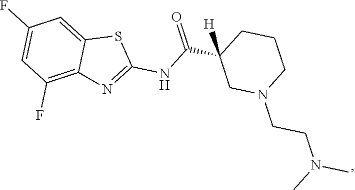 [SMILES: CN(C)CCN1CCC[C@@H](C(=O)Nc2nc3c(F)cc(F)cc3s2)C1]

or a pharmaceutically acceptable salt thereof.

In yet another general aspect, the present disclosure provides a pharmaceutical composition comprising(S)-N-(4,6-difluorobenzo[d]thiazol-2-yl)-1-(2-(dimethylamino)ethyl) piperidine-3-carboxamide, or a pharmaceutically acceptable salt thereof, and a pharmaceutically acceptable carrier.

In yet another general aspect, the present disclosure provides a pharmaceutical composition comprising (R)-N-(4,6-difluorobenzo[d]thiazol-2-yl)-1-(2-(dimethylamino)ethyl) piperidine-3-carboxamide, or a pharmaceutically acceptable salt thereof, and a pharmaceutically acceptable carrier.

In yet another general aspect, the present disclosure provides a method of modulating particulate guanylyl cyclase receptor A (pGC-A) in a cell, the method comprising contacting the cell with an effective amount of(S)-N-(4,6-difluorobenzo[d]thiazol-2-yl)-1-(2-(dimethylamino)ethyl) piperidine-3-carboxamide, or a pharmaceutically acceptable salt thereof.

In yet another general aspect, the present disclosure provides a method of modulating particulate guanylyl cyclase receptor A (pGC-A) in a cell, the method comprising contacting the cell with an effective amount of (R)-N-(4,6-difluorobenzo[d]thiazol-2-yl)-1-(2-(dimethylamino)ethyl) piperidine-3-carboxamide, or a pharmaceutically acceptable salt thereof.

In yet another general aspect, the present disclosure provides a method of modulating particulate guanylyl cyclase receptor A (pGC-A) in a subject, the method comprising administering to the subject in need thereof an effective amount of(S)-N-(4,6-difluorobenzo[d]thiazol-2-yl)-1-(2-(dimethylamino)ethyl) piperidine-3-carboxamide, or a pharmaceutically acceptable salt thereof, or the pharmaceutical composition comprising same.

In yet another general aspect, the present disclosure provides a method of modulating particulate guanylyl cyclase receptor A (pGC-A) in a subject, the method comprising administering to the subject in need thereof an effective amount of (R)-N-(4,6-difluorobenzo[d]thiazol-2-yl)-1-(2-(dimethylamino)ethyl) piperidine-3-carboxamide, or a pharmaceutically acceptable salt thereof, or the pharmaceutical composition comprising same.

In yet another general aspect, the present disclosure provides a method of treating or preventing a disease or condition responsive to modulation of a particulate guanylyl cyclase receptor A (pGC-A) in a subject, the method comprising administering to the subject in need thereof a therapeutically effective amount of(S)-N-(4,6-difluorobenzo[d]thiazol-2-yl)-1-(2-(dimethylamino)ethyl) piperidine-3-carboxamide, or a pharmaceutically acceptable salt thereof, or the pharmaceutical composition comprising same.

In yet another general aspect, the present disclosure provides a method of treating or preventing a disease or condition responsive to modulation of a particulate guanylyl cyclase receptor A (pGC-A) in a subject, the method comprising administering to the subject in need thereof a therapeutically effective amount of (R)-N-(4,6-difluorobenzo[d]thiazol-2-yl)-1-(2-(dimethylamino)ethyl) piperidine-3-carboxamide, or a pharmaceutically acceptable salt thereof, or the pharmaceutical composition comprising same.

In some embodiments, the disease or condition is selected from metabolic disease, cardiovascular disease, and kidney disease. Suitable examples of these diseases are described herein.

Unless otherwise defined, all technical and scientific terms used herein have the same meaning as commonly understood by one of ordinary skill in the art to which the present application belongs. Methods and materials are described herein for use in the present application: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the present application will be apparent from the following detailed description and figures, and from the claims.

### DESCRIPTION OF DRAWINGS

FIG. 1 depicts the liquid chromatography mass spectrometry (LCMS) trace for Compound 3 TFA salt.

FIG. 2 depicts the mass spectrometry (MS) spectrum for Compound 3 TFA salt.

FIG. 3 depicts the hydrogen-1 nuclear magnetic resonance (¹H NMR) for Compound 3 TFA salt.

FIG. 4 depicts the chiral liquid chromatography (LC) trace for Compound 3 TFA salt.

FIG. 5 depicts the chiral liquid chromatography (LC) trace for Compound 2 TFA salt.

FIG. 6 depicts the chiral liquid chromatography (LC) trace for Compound 1 TFA salt.

FIG. 7 depicts the liquid chromatography mass spectrometry (LCMS) trace for Compound 2 TFA salt.

FIG. 8 depicts the mass spectrometry (MS) spectrum for Compound 2 TFA salt.

FIG. 9 depicts the hydrogen-1 nuclear magnetic resonance (¹H NMR) for Compound 2 TFA salt.

FIG. 10 depicts the liquid chromatography mass spectrometry (LCMS) trace for Compound 1 TFA salt.

FIG. 11 depicts the mass spectrometry (MS) spectrum for Compound 1 TFA salt.

FIG. 12 depicts the hydrogen-1 nuclear magnetic resonance (¹H NMR) for Compound 1 TFA salt.

FIG. 13 depicts the liquid chromatography mass spectrometry (LCMS) trace for Compound 3 acetate salt.

FIG. 14 depicts the hydrogen-1 nuclear magnetic resonance (¹H NMR) for Compound 3 acetate salt.

FIG. 15 depicts the chiral liquid chromatography (LC) trace for Compound 2 acetate salt

FIG. 16 depicts the chiral liquid chromatography (LC) trace for Compound 1 acetate salt

FIG. 17 depicts the liquid chromatography mass spectrometry (LCMS) trace for Compound 2 acetate salt.

FIG. 18 depicts the mass spectrometry (MS) spectrum for Compound 2 acetate salt.

FIG. 19 depicts the hydrogen-1 nuclear magnetic resonance (¹H NMR) for Compound 2 acetate salt.

FIG. 20 depicts the liquid chromatography mass spectrometry (LCMS) trace for Compound 1 acetate salt.

FIG. 21 depicts the mass spectrometry (MS) spectrum for Compound 1 acetate salt.

FIG. 22 depicts the hydrogen-1 nuclear magnetic resonance (¹H NMR) for Compound 1 acetate salt.

FIG. 23 is a bar graph of cGMP production levels in HEK293 cell overexpressing human GC-A receptor induced by vehicle and Compound 3 acetate salt.

FIG. 24 is a bar graph of cGMP production levels in HEK293 cell overexpressing human GC-A receptor induced by vehicle and Compound 3 acetate salt in the presence of ANP (10-10 M).

FIG. 25 is a bar graph of cGMP production levels in primary human cardiomyocytes induced by vehicle and Compound 3 acetate salt in the presence of ANP (10-10 M).

FIG. 26 is a comparison of change in plasma cGMP level induced by an IV bolus administration of vehicle or 10 mg/kg IV bolus of Compound 3 acetate salt in spontaneously hypertensive rats.

FIG. 27 is a comparison of change in urinary cGMP level induced by an IV bolus administration of vehicle or 10 mg/kg IV bolus of Compound 3 acetate salt in spontaneously hypertensive rats.

FIG. 28 is a comparison of a change in systolic blood pressure reduction induced by an IV bolus administration of vehicle or 10 mg/kg IV bolus of Compound 3 acetate salt in spontaneously hypertensive rats.

FIG. 29 is a comparison of a change in diastolic blood pressure reduction induced by an IV bolus administration of vehicle or 10 mg/kg IV bolus of Compound 3 acetate salt in spontaneously hypertensive rats.

FIG. 30 is a bar graph of urinary volume before (pre-bolus) and after (post-bolus) an IV bolus administration of vehicle or 10 mg/kg IV bolus of Compound 3 acetate salt in spontaneously hypertensive rats.

FIG. 31 is a bar graph of urinary sodium excretion rate before (pre-bolus) and after (post-bolus) an IV bolus administration of vehicle or 10 mg/kg IV bolus of Compound 3 acetate salt in spontaneously hypertensive rats.

FIG. 32 is a bar graph of cGMP production levels in HEK293 cell overexpressing human GC-A receptor induced by vehicle, Compound 1 or Compound 2 acetate salt.

FIG. 33 is a bar graph of cGMP production levels in HEK293 cell overexpressing human GC-A receptor induced by vehicle, Compound 1 or Compound 2 acetate salt in the presence of ANP (10-10 M).

FIG. 34 is a bar graph of cGMP production levels in primary human cardiomyocytes induced by vehicle, Compound 1 or Compound 2 acetate salt in the presence of ANP (10-10 M).

FIG. 35 is a comparison of change in plasma cGMP level induced by an IV bolus administration of vehicle, 10 mg/kg IV bolus of Compound 2 or 10 mg/kg IV bolus of Compound 1 acetate salt in spontaneously hypertensive rats.

FIG. 36 is a comparison of change in urinary cGMP level induced by an IV bolus administration of vehicle, 10 mg/kg IV bolus of Compound 2 or 10 mg/kg IV bolus of Compound 1 acetate salt in spontaneously hypertensive rats.

FIG. 37 is a comparison of a change in systolic blood pressure reduction induced by an IV bolus administration of vehicle, 10 mg/kg IV bolus of Compound 2 or 10 mg/kg IV bolus of Compound 1 acetate salt in spontaneously hypertensive rats.

FIG. 38 is a comparison of a change in diastolic blood pressure reduction induced by an IV bolus administration of vehicle, 10 mg/kg IV bolus of Compound 2 or 10 mg/kg IV bolus of Compound 1 acetate salt in spontaneously hypertensive rats.

FIG. 39 is a bar graph of urinary volume before (pre-bolus) and after (post-bolus) an IV bolus administration of vehicle, 10 mg/kg IV bolus of Compound 2 or 10 mg/kg IV bolus of Compound 1 acetate salt in spontaneously hypertensive rats.

FIG. 40 is a bar graph of urinary sodium excretion rate before (pre-bolus) and after (post-bolus) an IV bolus administration of vehicle, 10 mg/kg IV bolus of Compound 2 or 10 mg/kg IV bolus of Compound 1 acetate salt in spontaneously hypertensive rats.

FIG. 41 is a schematic representation showing pGC-A receptor, to which ANP and BNP bind, possesses pleiotropic actions via cGMP generation that leads to a therapeutic effect for cardiovascular, renal and metabolic disease, as well as cancer.

FIG. 42 is a line plot showing concentration-response curves for cGMP response of(S)-N-(4,6-difluorobenzo[d]thiazol-2-yl)-1-(2-(dimethylamino)ethyl) piperidine-3-carboxamide (compound 1) (squares) and of (R)-N-(4,6-difluorobenzo[d]thiazol-2-yl)-1-(2-(dimethylamino)ethyl) piperidine-3-carboxamide (compound 2, free base) (circles) in HEK 293 human pGC-A expressing cells in the presence of ANP. Data is average of 3 independent experiments.

### DETAILED DESCRIPTION

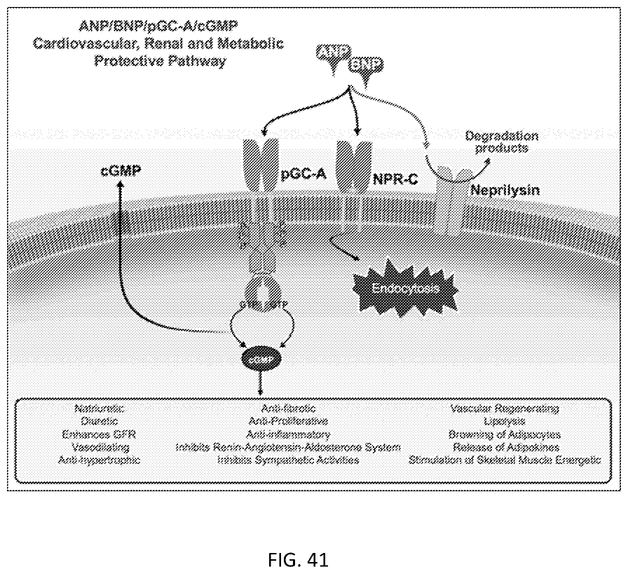

Without being bound by a particular theory; it is believed that the heart is a vital endocrine organ that fine-tunes the body's cardiovascular and metabolic homeostasis. Atrial natriuretic peptide (ANP) and B-type natriuretic peptide (BNP) are produced in the heart and released from atrial secretory granules, much like insulin is produced and released from pancreatic secretory granules. The molecular target of these two cardiac hormones is the particulate guanylyl cyclase receptor A (pGC-A) (See FIG. 41) which functions via the second messenger cGMP. Among various physiological functions of pGC-A are regulation of blood pressure (BP), reno-enhancing and renoprotective actions, as well as metabolic actions, including lipolysis with production of non-esterified free fatty acids (NEFA) and glycerol, browning of white adipocytes, stimulation of skeletal muscle energetics, and enhancing release of adipokines such as adiponectin. In one example, in a murine model of obesity; over-expression of the pGC-A activating cardiac hormone BNP protected animals from obesity. In addition, pGC-A is highly expressed in the heart, kidney, adrenals, vasculature, and adipocytes. While optimally regulating intravascular volume and blood pressure homeostasis, pGC-A activation directly mediates organ protection with anti-apoptotic, anti-fibrotic, anti-hypertrophic, vascular endothelial regenerating, lipolytic, aldosterone suppressing, anti-cancer, and tumor suppressive properties.

Population studies investigating the common genetic variants of the ANP (rs5068) and BNP genes (rs1938845) showed that rs5068 and rs1938845 increase circulating ANP or BNP, respectively. Importantly, the elevation of ANP, through rs5068, was associated with protection from obesity and metabolic syndrome, decreased waist circumference, higher HDL levels with reduced BP, and risk for hypertension. While rs5068 is common, only 10% of the population carry this ANP genetic variant, and exhibit the protective phenotype. Hence, approximately 90% of the population has a relative higher risk for metabolic syndrome and hypertension based upon this ANP genetic variation. It was also shown that metabolic protective actions of rs5068 are present in African Americans, which highlights the multiethnic metabolic protection of pGC-A. Furthermore, the BNP gene variant, rs1938845 was found to be associated with reduced risk for type II diabetes mellitus as well as prolonged survival of the diabetes patients. Importantly, in patients with chronic heart failure, twice daily subcutaneously (SQ) administered BNP and subsequently pGC-A activation reversed cardiac hypertrophy and improved myocardial function and notably, improved patient symptoms.

Without being bound by a theory, it is believed that the compounds described herein increase pGC-A responsiveness to the endogenous ligands (ANP and BNP), even at reduced levels, by enhancing the pGC-A function in a positive allosteric manner. The compounds within the present claims also exhibited good ADME (Absorption, Distribution, Metabolism, and Excretion) properties including solubility, microsomal stability and plasma stability.

### Therapeutic Compounds

In one general aspect, the present disclosure provides a compound(S)-N-(4,6-difluorobenzo[d]thiazol-2-yl)-1-(2-(dimethylamino)ethyl) piperidine-3-carboxamide having formula:

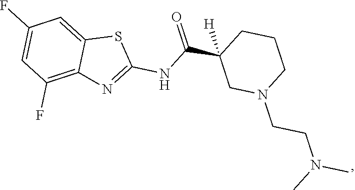 [SMILES: CN(C)CCN1CCC[C@H](C(=O)Nc2nc3c(F)cc(F)cc3s2)C1]

or a pharmaceutically acceptable salt thereof.

In another general aspect, the present disclosure provides a compound (R)-N-(4,6-difluorobenzo[d]thiazol-2-yl)-1-(2-(dimethylamino)ethyl) piperidine-3-carboxamide having formula:

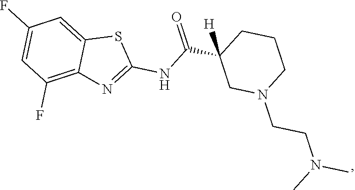 [SMILES: CN(C)CCN1CCC[C@@H](C(=O)Nc2nc3c(F)cc(F)cc3s2)C1]

or a pharmaceutically acceptable salt thereof.

### Pharmaceutically Acceptable Salts

In some embodiments, a salt of a compound of the instant disclosure is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of the present disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, capry late, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxy benzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesul fonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of the present disclosure include hydroxides of alkali metals, including sodium, potassium, and lithium: hydroxides of alkaline earth metals such as calcium and magnesium: hydroxides of other metals, such as aluminum and zinc: ammonia, organic amines such as unsubstituted or hydroxyl-substituted mono-, di-, or tri-alkylamines, dicyclohexylamine: tributyl amine: pyridine: N-methyl, N-ethylamine: diethylamine; triethylamine: mono-, bis-, or tris-(2-OH—(C₁-C₆)-alkylamine), such as N,N-dimethyl-N-(2-hydroxyethyl)amine or tri-(2-hydroxyethyl)amine: N-methyl-D-glucamine: morpholine: thiomorpholine: piperidine: pyrrolidine; and amino acids such as arginine, lysine, and the like. In some embodiments, the compounds of Formula (I), or pharmaceutically acceptable salts thereof, are substantially isolated.

### Methods of Making Therapeutic Compounds

Compounds of this disclosure, including salts thereof, can be prepared using known organic synthesis techniques and can be synthesized according to any of numerous possible synthetic routes. A person skilled in the art knows how to select and implement appropriate synthetic protocols, and appreciates that the processes described are not the exclusive means by which compounds provided herein may be synthesized, and that a broad repertoire of synthetic organic reactions is available to be potentially employed in synthesizing compounds provided herein.

Suitable synthetic methods of starting materials, intermediates and products may be identified by reference to the literature, including reference sources such as: Advances in Heterocyclic Chemistry, Vols. 1-107 (Elsevier, 1963-2012): Journal of Heterocyclic Chemistry Vols. 1-49 (Journal of Heterocyclic Chemistry, 1964-2012): Carreira, et al. (Ed.) Science of Synthesis, Vols. 1-48 (2001-2010) and Knowledge Updates KU2010/1-4: 2011/1-4: 2012/1-2 (Thieme, 2001-2012): Katritzky, et al. (Ed.) Comprehensive Organic Functional Group Transformations, (Pergamon Press, 1996): Katritzky et al. (Ed.): Comprehensive Organic Functional Group Transformations II (Elsevier, 2^{nd} Edition, 2004): Katritzky et al. (Ed.), Comprehensive Heterocyclic Chemistry (Pergamon Press, 1984): Katritzky et al., Comprehensive Heterocyclic Chemistry II, (Pergamon Press, 1996): Smith et al., March's Advanced Organic Chemistry: Reactions. Mechanisms, and Structure, 6th Ed. (Wiley, 2007); Trost et al. (Ed.), Comprehensive Organic Synthesis (Pergamon Press, 1991).

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4th Ed., Wiley & Sons, Inc., New York (2006).

### Methods of Using Therapeutic Compounds

The present disclosure provides, at least in part, that the pGC-A/cGMP pathway is a valuable molecular target for metabolic, cardiovascular (CV), renal, and anti-cancer therapeutics. As discussed above, the elevation of pGC-A's endogenous ligand ANP levels is associated with protection from obesity and metabolic syndrome, reduced blood pressure, decreased risk for hypertension as well as reduced incidence of myocardial infarction. Similarly, the elevation of levels of endogenous ligand BNP is associated with reduced risk for type II diabetes mellitus.

Accordingly, in a general aspect, the present disclosure provides a method of modulating particulate guanylyl cyclase receptor A (pGC-A) in a cell, the method comprising contacting the cell with an effective amount of a compound as described herein, or a pharmaceutically acceptable salt thereof. In some embodiments, the cell is contacted in vitro, in vivo, or ex vivo.

The present disclosure also provides a method of modulating particulate guanylyl cyclase receptor A (pGC-A) in a subject, the method comprising administering to the subject in need thereof an effective amount of a compound as described herein, or a pharmaceutically acceptable salt thereof, or the pharmaceutical composition comprising same.

In some embodiments of the methods of the present disclosure, modulating of the particulate guanylyl cyclase receptor A (pGC-A) comprises positive allosteric enhancement of activity of the particulate guanylyl cyclase receptor A (pGC-A) (e.g., the modulating comprises increased production cGMP in a cell (e.g., in a cell of the subject). In some embodiments, the cell is a renal cell or a heart muscle cell.

The present disclosure also provides a method of treating or preventing a disease or condition responsive to modulation of a particulate guanylyl cyclase receptor A (pGC-A) in a subject, the method comprising administering to the subject in need thereof a therapeutically effective amount of a compound of the present disclosure, or a pharmaceutically acceptable salt thereof, or the pharmaceutical composition comprising same.

The present disclosure also provides(S)-N-(4,6-difluorobenzo[d]thiazol-2-yl)-1-(2-(dimethylamino)ethyl) piperidine-3-carboxamide, or a pharmaceutically acceptable salt thereof, or the pharmaceutical composition comprising same, for use in a manufacture of a medicament for the treatment or prevention of a disease or condition responsive to modulation of a particulate guanylyl cyclase receptor A (pGC-A) in a subject.

The present disclosure also provides (R)-N-(4,6-difluorobenzo[d]thiazol-2-yl)-1-(2-(dimethylamino)ethyl) piperidine-3-carboxamide, or a pharmaceutically acceptable salt thereof, or the pharmaceutical composition comprising same, for use in a manufacture of a medicament for the treatment or prevention of a disease or condition responsive to modulation of a particulate guanylyl cyclase receptor A (pGC-A) in a subject.

The present disclosure also provides(S)-N-(4,6-difluorobenzo[d]thiazol-2-yl)-1-(2-(dimethylamino)ethyl) piperidine-3-carboxamide, or a pharmaceutically acceptable salt thereof, or the pharmaceutical composition comprising same, for use the treatment or prevention of a disease or condition responsive to modulation of a particulate guanylyl cyclase receptor A (pGC-A) in a subject.

The present disclosure also provides (R)-N-(4,6-difluorobenzo[d]thiazol-2-yl)-1-(2-(dimethylamino)ethyl) piperidine-3-carboxamide, or a pharmaceutically acceptable salt thereof, or the pharmaceutical composition comprising same, for use the treatment or prevention of a disease or condition responsive to modulation of a particulate guanylyl cyclase receptor A (pGC-A) in a subject.

In some embodiments, the disease or condition responsive to modulation of a particulate guanylyl cyclase receptor A (pGC-A) is a metabolic disease or disorder. In some embodiments, the metabolic disorder is congenital. Suitable examples of such disorders include Fabry disease, phenylketonuria, Prader-Willi syndrome, galactosemia, Tay-Sachs's disease, porphyria, Pompe disease, Neimann-Pick disease, Morquio's syndrome, Morteaus-lamy syndrome, Hunter syndrome, Lesh-Nyhan syndrome, Hurler syndrome, homocystinuria,

Hartnup disease, and Gaucher's disease. In some embodiments, the metabolic disorder is acquired. Suitable examples of such disorders include diabetes (e.g., type 1 diabetes, diabetes insipidus, or type II diabetes mellitus), obesity; metabolic syndrome, dyslipidemia, hipolipidemia (hyperlipoproteinemia), hyperthyroidism, hypoparathyroidism, hypothyroidism, Cushing's syndrome, hyperuricemia, hemochromatosis, and hyperparathyroidism. Other examples of metabolic disorders include glucose intolerance, insulin resistance, fibrinolysis disorder, endothelial dysfunction, atherosclerosis, impaired fasting glycemia, hyperinsulinemia, galactosemia, mucopolysaccaridose, tyrosinemia, methylmalonic aciduria, acidemia (e.g., propionic acidemia, isovaleric acidemia), and hyperammonemia. In some embodiments, the metabolic disease is selected from obesity; hypertriglyceridemia, metabolic syndrome, insulin resistance, hyperinsulinemia, diabetes, and acidemia.

In some embodiments, the disease or condition responsive to modulation of a particulate guanylyl cyclase receptor A (pGC-A) is a cardiovascular disease. Suitable examples of cardiovascular disorders include high blood pressure, myocardial infarction, abnormal heart rhythms (e.g., arrhythmia), aorta disease, Marfan syndrome, congenital heart disease, coronary artery disease (e.g., narrowing of the arteries), deep vein thrombosis, pulmonary embolism, heart attack, heart failure, heart muscle disease (e.g., cardiomyopathy), heart valve disease, pericardial disease, peripheral vascular disease, rheumatic heart disease, stroke, vascular disease (e.g., blood vessel disease), cardiomyopathies, hypertension, aortic stenosis, mitral valve insufficiency, mitral valve prolapse, pericarditis, rheumatic heart disease, and cardiorenal syndrome. In some embodiments, the cardiovascular disease is selected from heart failure, cardiomyopathy; hypertension, high blood pressure, and myocardial infarction.

In some embodiments, the disease or condition responsive to modulation of a particulate guanylyl cyclase receptor A (pGC-A) is kidney disease. Suitable examples of renal diseases include nephropathy; acute kidney injury; kidney failure, acute renal failure, kidney stones, glomerulonephritis, polycystic kidney disease, urinary tract infections, kidney infection (pyelonephritis), simple kidney cysts, diabetic kidney disease, nephropathy; lupus nephritis, Henoch-Schönlein purpura, goodpasture syndrome, ectopic kidney, amyloidosis, acquired cystic kidney disease, glomerular disease, kidney dysplasia, medullary sponge kidney, nephrotic syndrome, kidney damage, renal artery stenosis, renal tubular acidosis, and solitary kidney: In some embodiments, the kidney disease is selected from nephropathy; acute renal failure, chronic kidney disease, cardiorenal syndrome and diabetic kidney disease.

In some embodiments, the disease or condition responsive to modulation of a particulate guanylyl cyclase receptor A (pGC-A) is cancer. Suitable example of cancer include bladder cancer, brain cancer, breast cancer, colorectal cancer (e.g., colon cancer), rectal cancer, cervical cancer, gastrointestinal cancer, genitourinary cancer, head and neck cancer, lung cancer, oral cancer, ovarian cancer, pancreatic cancer (e.g., pancreatic neuroendocrine tumor), prostate cancer, endometrial cancer, renal cancer (kidney cancer) (e.g., advanced kidney cancer), skin cancer, liver cancer, thyroid cancer, leukemia, and testicular cancer.

### Pharmaceutical Compositions and Formulations

The present application also provides pharmaceutical compositions comprising an effective amount of(S)- or (R)-N-(4,6-difluorobenzo[d]thiazol-2-yl)-1-(2-(dimethylamino)ethyl) piperidine-3-carboxamide, or a pharmaceutically acceptable salt thereof, and a pharmaceutically acceptable carrier. The pharmaceutical composition may also comprise any one of the additional therapeutic agents described herein, or a pharmaceutically acceptable salt thereof. In certain embodiments, the application also provides pharmaceutical compositions and dosage forms comprising any one the additional therapeutic agents described herein, or a pharmaceutically acceptable salt thereof, and a pharmaceutically acceptable carrier. The carrier(s) and excipient(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary; intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy; Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary; shaping the product.

In some embodiments, any one of the compounds and therapeutic agents disclosed herein are administered orally: Compositions of the present application suitable for oral administration may be presented as discrete units such as capsules, sachets, granules or tablets each containing a predetermined amount (e.g., effective amount) of the active ingredient: a powder or granules: a solution or a suspension in an aqueous liquid or a non-aqueous liquid: an oil-in-water liquid emulsion: a water-in-oil liquid emulsion: packed in liposomes: or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray; stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562:5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, (S) or (R)-N-(4,6-difluorobenzo[d]thiazol-2-yl)-1-(2-(dimethylamino)ethyl) piperidine-3-carboxamides present in an effective amount (e.g., a therapeutically effective amount). Effective doses may vary; depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a compound disclosed herein can range, for example, from about 0.001 mg/kg to about 500 mg/kg (e.g., from about 0.001 mg/kg to about 200 mg/kg: from about 0.01 mg/kg to about 200 mg/kg: from about 0.01 mg/kg to about 150 mg/kg: from about 0.01 mg/kg to about 100 mg/kg: from about 0.01 mg/kg to about 50 mg/kg: from about 0.01 mg/kg to about 10 mg/kg: from about 0.01 mg/kg to about 5 mg/kg: from about 0.01 mg/kg to about 1 mg/kg: from about 0.01 mg/kg to about 0.5 mg/kg: from about 0.01 mg/kg to about 0.1 mg/kg: from about 0.1 mg/kg to about 200 mg/kg: from about 0.1 mg/kg to about 150 mg/kg: from about 0.1 mg/kg to about 100 mg/kg: from about 0.1 mg/kg to about 50 mg/kg: from about 0.1 mg/kg to about 10 mg/kg: from about 0.1 mg/kg to about 5 mg/kg: from about 0.1 mg/kg to about 2 mg/kg: from about 0.1 mg/kg to about 1 mg/kg: or from about 0.1 mg/kg to about 0.5 mg/kg). In some embodiments, an effective amount of a compound disclosed herein is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent in a suitable amount or dosage.

### Definitions

As used herein, the term “about” means “approximately” (e.g., plus or minus approximately 10% of the indicated value).

As used herein, the term “compound” as used herein is meant to include all stereoisomers, geometric isomers, tautomers, and isotopes of the structures named or depicted. Compounds herein identified by name or structure as one particular tautomeric form are intended to include other tautomeric forms unless otherwise specified. The term “compound,” when referring to a compound of this disclosure, refers to a collection of molecules (at least two molecules) having an identical chemical structure (the term encompasses more than a single molecule). In some embodiments, the present disclosure provides a compound N-(4,6-difluorobenzo[d]thiazol-2-yl)-1-(2-(dimethylamino)ethyl) piperidine-3-carboxamide having formula:

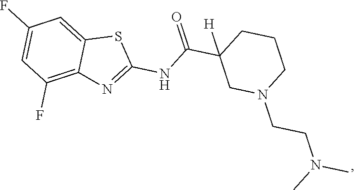 [SMILES: CN(C)CCN1CCCC(C(=O)Nc2nc3c(F)cc(F)cc3s2)C1]

and it is understood that this compound encompasses a racemic mixture of(S)-N-(4,6-difluorobenzo[d]thiazol-2-yl)-1-(2-(dimethylamino)ethyl) piperidine-3-carboxamide and (R)-N-(4,6-difluorobenzo[d]thiazol-2-yl)-1-(2-(dimethylamino)ethyl) piperidine-3-carboxamide. That is, the above-depicted compound encompasses a collection of molecules containing the R- and the S-isomer in equal proportions (a 50/50 mixture). For example, the above-depicted compound encompasses a collection of one molecule of R-isomer and one molecule of S-isomer. In another example, the compound(S)-N-(4,6-difluorobenzo[d]thiazol-2-yl)-1-(2-(dimethylamino)ethyl) piperidine-3-carboxamide, having formula:

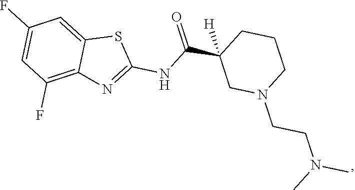 [SMILES: CN(C)CCN1CCC[C@H](C(=O)Nc2nc3c(F)cc(F)cc3s2)C1]

encompasses a collection of at least two molecules that are both S-isomers as depicted.

As used herein, the term “tautomer” refers to compounds which are capable of existing in a state of equilibrium between two isomeric forms. Such compounds may differ in the bond connecting two atoms or groups and the position of these atoms or groups in the compound.

As used herein, the term “isomer” refers to structural, geometric and stereo isomers. The terms “pharmaceutical” and “pharmaceutically acceptable” are employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system, an in vivo system, or an ex vivo system. For example, “contacting” the particulate guanylyl cyclase receptor A with a compound of the invention includes the administration of a compound of the present invention to an individual or patient, such as a human, having particulate guanylyl cyclase receptor A, as well as, for example, introducing a compound of the invention into a sample containing a cellular or purified preparation containing the particulate guanylyl cyclase receptor A.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease: for example, inhibiting a disease, condition or disorder in an individual who is experiencing or displaying the pathology or symptomatology of the disease, condition or disorder (i.e., arresting further development of the pathology and/or symptomatology), or 2) ameliorating the disease: for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

### Example 1

Test compounds at 10 mM DMSO stock concentration were added to 384 well assay plates using Tecan dispenser starting at 40 μM concentration and diluted 2-fold for 10-point concentration-response curves. Wells were backfilled with DMSO such that the final concentration of DMSO in all wells was maintained at 0.3% DMSO. Positive allosteric modulation (presence of EC₃₀ concentration of ANP at 1.3 pM) of pGC-A-mediated activation of cGMP production was measured in human pGC-A overexpressing HEK293 cells by homogenous time-resolved florescence (HTRF) competition assay using cGMP CisBio detection kit (Cat #: 62GM2PEC). Enantiomerically pure(S)-N-(4,6-difluorobenzo[d]thiazol-2-yl)-1-(2-(dimethylamino)ethyl) piperidine-3-carboxamide and (R)-N-(4,6-difluorobenzo[d]thiazol-2-yl)-1-(2-(dimethylamino)ethyl) piperidine-3-carboxamide were individually synthesized from enantiomerically pure starting materials (R- and S-nipecotic acid).

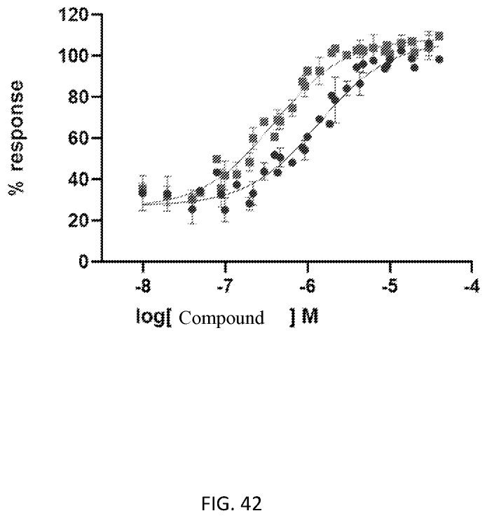

(S)-N-(4,6-difluorobenzo[d]thiazol-2-yl)-1-(2-(dimethylamino)ethyl) piperidine-3-carboxamide as the free base (referred herein as compound 1) was similar in potency to the racemic mixture (referred herein as compound 3) and 3.5-fold more potent than the R-enantiomer as the free base (referred herein as compound 2) (pEC₅₀=6.4+0.2 and 5.9+0.2, respectively). Concentration-response curves for both tested compounds in HEK 293 human pGC-A expressing cells in the presence of ANP is shown in FIG. 42.

### Example 2—Preparation of Trifluoroacetate Salt of Compound 3

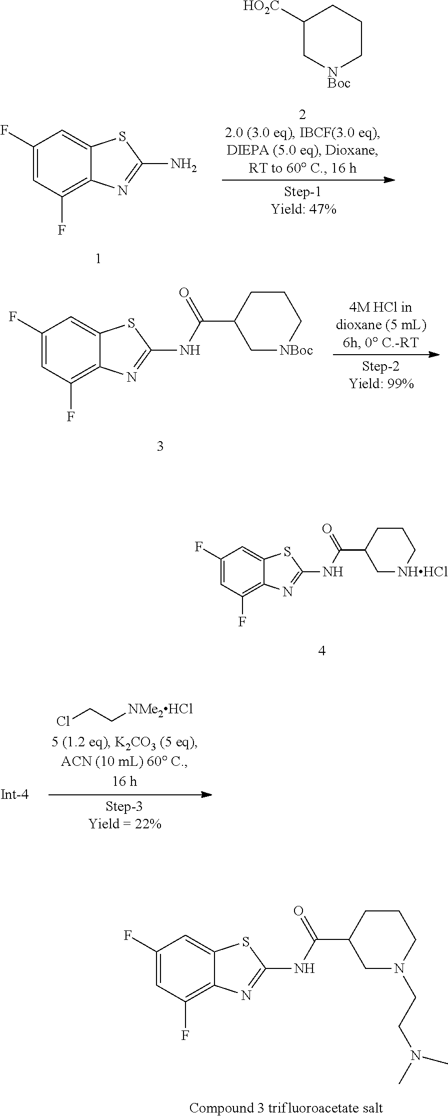

Step-1: Synthesis of tert-butyl 3-((4.6-difluorobenzo[d]thiazol-2-yl) carbamoyl) piperidine-1-carboxylate: IBCF (4.4 g, 32.2 mmol) was added to a mixture of 1-[(tert-butoxy) carbonyl]piperidine-3-carboxylic acid (7.39 g, 32.2 mmol) and DIPEA (9.9 mL, 53.7 mmol) in 1,4-dioxane (60 mL) at RT and resulted mixture was stirred for 15 min. Then 4,6-difluoro-1,3-benzothiazol-2-amine (2 g, 10.7 mmol) was added to the solution, the reaction mixture was stirred at 60° C. for 16 h. After completion of the reaction (monitored by TLC and LCMS), reaction mixture was diluted with water (50 mL) and extracted with ethyl acetate (100 mL×2). The organic layer was dried over Na₂SO₄ and concentrated under reduced pressure to get crude. The crude material was purified by flash chromatography on silica gel to eluent (20% EtOAc in hexane) to obtain the desired compound tert-butyl 3-((4, 6-difluorobenzo[d]thiazol-2-yl) carbamoyl) piperidine-1-carboxylate (2.0 g, 47% yield) as a colorless gum. ¹H NMR (DMSO-d₆, 400 MHZ) δ:12.74 (bs, 1H), 7.79 (d, J=7.2 Hz, 1H), 7.31 (t, J=9.25, 1H), 4.10-3.60 (m, 2H), 3.05-2.85 (m, 2H), 2.20-2.10 (m, 2H), 2.05-1.90 (m, 1H), 1.80-1.60 (m, 2H), 1.36 (s, 9H).

Step-2: Synthesis of N-(4.6-difluorobenzo[d]thiazol-2-yl) piperidine-3-carboxamide hydrochloride: To an oven dry round bottom flask tert-butyl 3-[(4,6-difluoro-1,3-benzothiazol-2-yl) carbamoyl]piperidine-1-carboxylate (2.0 g, 5.0 mmol) in DCM (20 mL) was added 4M HCl in dioxane (10 mL) at 0° C., then stirred at RT for 6 h. After completion, the reaction mixture was concentrated to get residue, then triturated with ether to get desired compound N-(4,6-difluorobenzo[d]thiazol-2-yl) piperidine-3-carboxamide hydrochloride (1.7 g, 99% yield) as a white solid. ¹H NMR (DMSO-d₆, 400 MHZ) δ:12.89 (bs, 1H), 8.93 (bs, 3H), 7.81 (d, J=8.8 Hz, 1H), 7.38 (t, J=11.2, 1H), 3.25-3.05 (m, 3H), 3.0-2.82 (m, 1H), 2.12-2.05 (m, 1H), 1.90-1.78 (m, 1H), 1.76-1.58 (m, 3H).

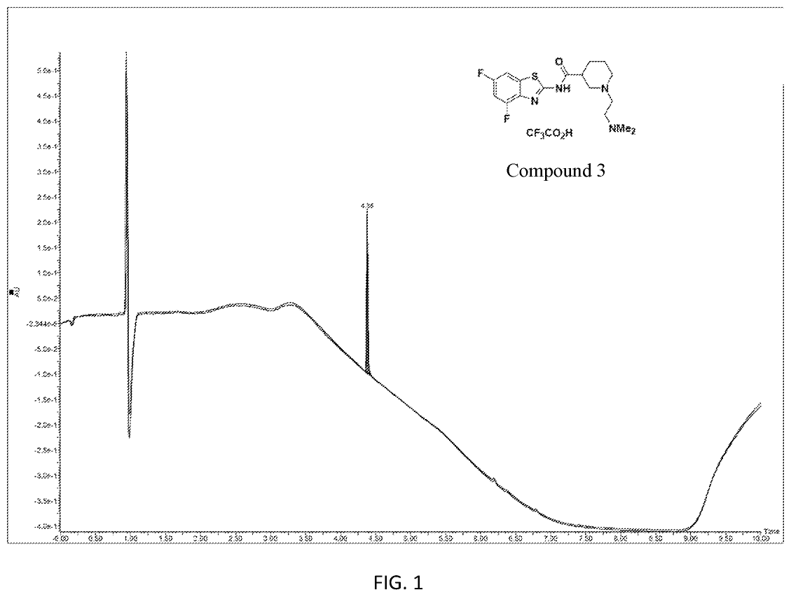

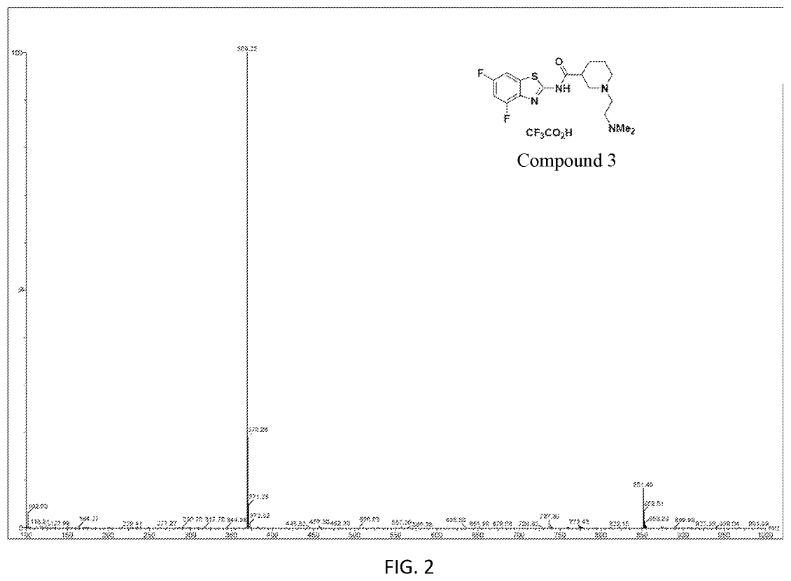

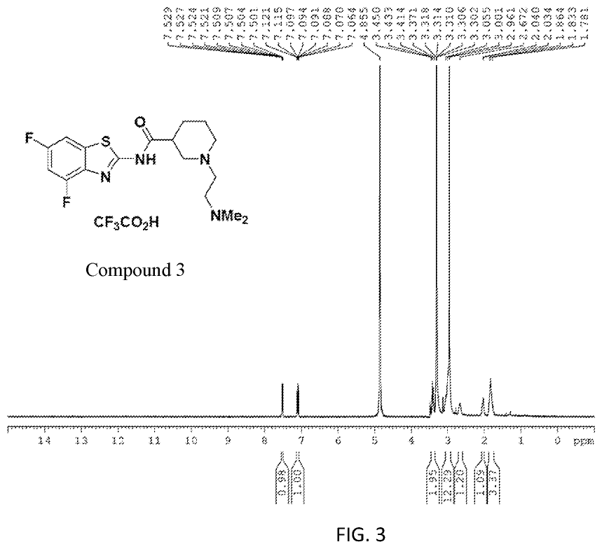

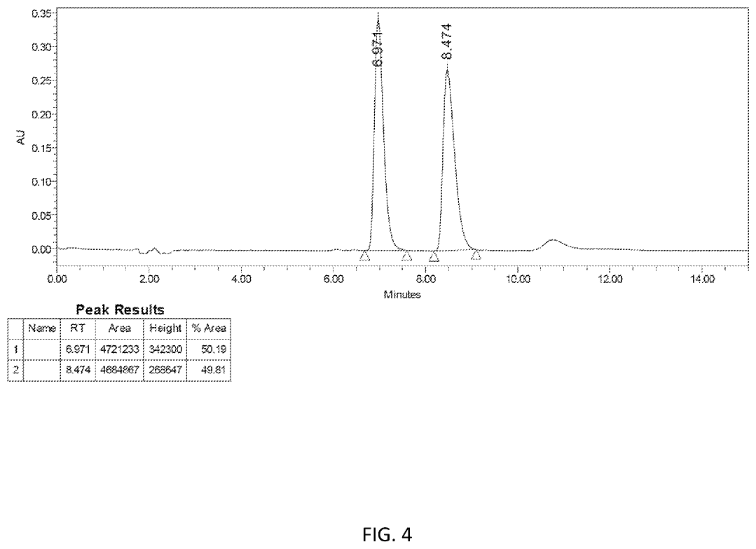

Step-3: Synthesis of N-(4.6-difluorobenzo[d]thiazol-2-yl)-1-(2-(dimethylamino)ethyl) piperidine-3-carboxamide: 2-chloro-N,N-dimethylethan-1-amine hydrochloride (774 mg, 7.19 mmol) was added to the stirred suspension of N-(4,6-difluoro-1,3-benzothiazol-2-yl) piperidine-3-carboxamide hydrochloride (1 g, 2.98 mmol) and K₂CO₃ (1.65 g, 12 mmol) in acetonitrile (25 mL) at room temperature. Resulting mixture was stirred at 60° C. overnight and the progress of reaction was monitored by TLC and LCMS. Reaction mixture was concentrated under vacuum to get crude. The crude was purified by prep HPLC to obtain the desired compound TFA salt of N-(4,6-difluorobenzo[d]thiazol-2-yl)-1-(2-(dimethylamino)ethyl) piperidine-3-carboxamide (497 mg, 22.5%) as a white solid. LCMS Calculated: m/z 368.45. LCMS Observed: m/z 369.22 [M+H]⁺. LCMS chromatogram is provided in FIG. 1. MS result is provided in FIG. 2. ¹H NMR is provided in FIG. 3. Chiral LC chromatogram is provided in FIG. 4. ¹H NMR (CD₃OD, 400 MHZ) 8:7.53-7.49 (m, 1H), 7.12-7.06 (m, 1H), 3.46-3.37 (m, 2H), 3.10-2.85 (m, 12H), 2.73-2.60 (m, 1H), 2.10-1.98 (m, 1H), 1.90-1.77 (m, 3H). Prep purification method: Column: X BRIDGE SHIELD (19×250) mm, 10 μm. Mobile phase: A, 0.1% TFA in water; B, ACN. Flow mode: 18 ml/min. Sample preparation diluents: ACN, H₂O. Wavelength: 214 nm.

### Example 3—Preparation of Acetate Salt of Compound 3

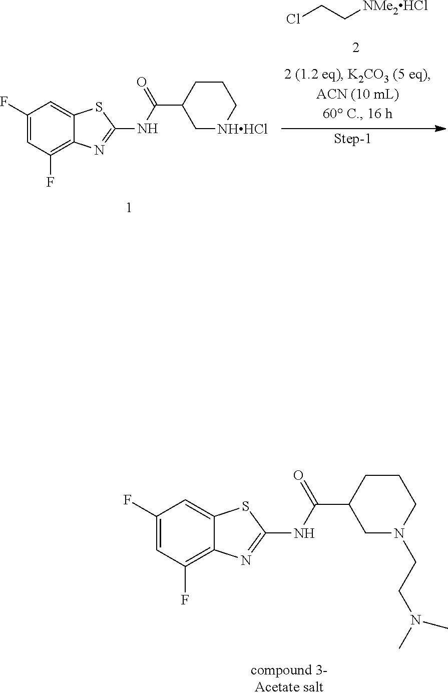

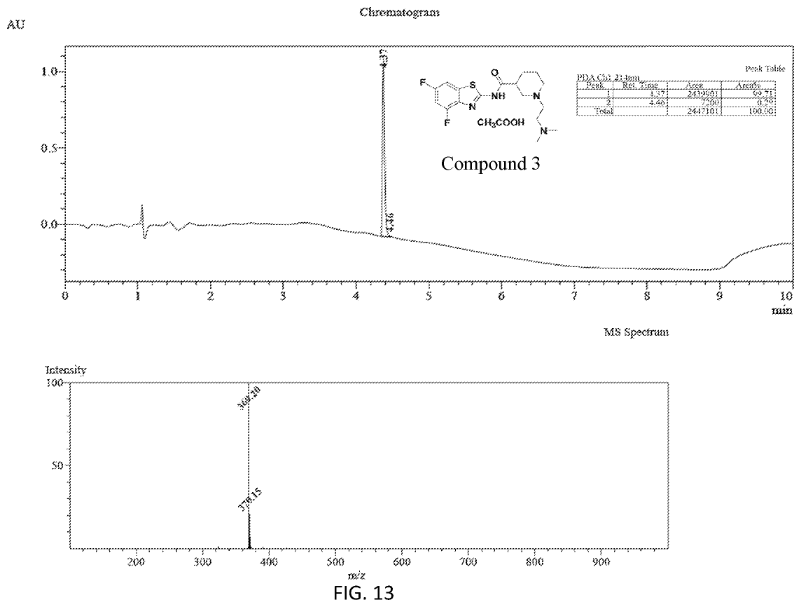

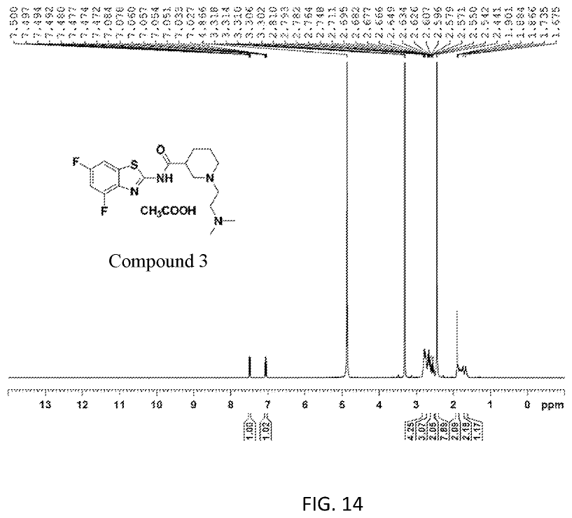

Synthesis of N-(4.6-difluorobenzo[d]thiazol-2-yl)-1-(2-(dimethylamino)ethyl) piperidine-3-carboxamide: 2-chloro-N,N-dimethylethan-1-amine hydrochloride (407 mg, 2.83 mmol) was added to the stirred suspension of N-(4,6-difluoro-1,3-benzothiazol-2-yl) piperidine-3-carboxamide (prepared as in Example 2 above) (0.7 g, 2.35 mmol) and K₂CO₃ (1.63 g, 11.8 mmol) in acetonitrile (10 mL) at room temperature. Resulting mixture was stirred at 60° C. for 16 h. The progress of reaction was monitored by TLC and LCMS. Reaction mixture was concentrated under vacuum to get crude. The crude was purified by prep-HPLC using 5 mM ammonium acetate in water as buffer to obtain the desired compound acetate salt of N-(4,6-difluoro-1,3-benzothiazol-2-yl)-1-[2-(dimethylamino)ethyl]piperidine-3-carboxamide (150 mg, 17% yield) as a white solid. LCMS Calculated: m/z 368.45. LCMS Observed: m/z 369.15 [M+H]⁺. LCMS chromatogram is shown in FIG. 13. ¹H NMR spectrum is shown in FIG. 14. ¹H NMR (CD₃OD, 400 MHZ) δ:7.51-7.48 (m, 1H), 7.09-7.04 (m, 1H), 3.03-2.98 (m, 1H), 2.98-2.79 (m, 3H), 2.75-2.66 (m, 3H), 2.64-2.58 (m, 7H), 2.50-2.42 (m, 1H), 1.95-1.88 (m, 3H), 1.86-1.67 (m, 3H). Prep purification method: Column: X SUNFIRE C18 (19×250) mm, 10 μm. Mobile phase: A, 5 mM Ammonium acetate in Water: B, ACN. Flow mode: 18 ml/min. Sample preparation diluents: ACN, H₂O. Wavelength: 214 nm.

### Example 4—Preparation of Trifluoroacetate Salt of Compound 1

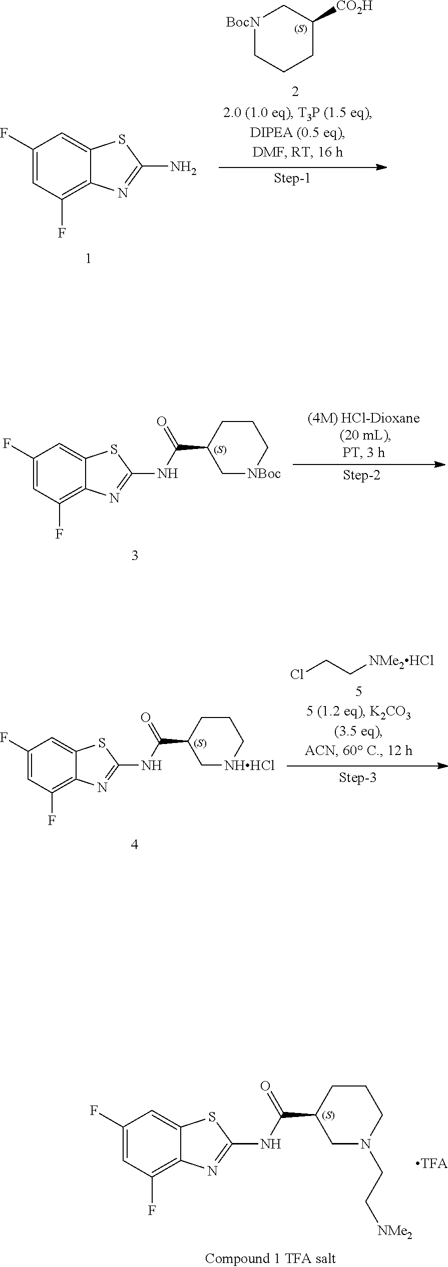

Step-1: Synthesis of tert-butyl(S)-3-(4.6-difluorobenzo[d]thiazol-2-yl) carbamoyl) piperidine-1-carboxylate: To a stirred solution of 4,6-difluoro-1,3-benzothiazol-2-amine (1 g, 5.35 mmol) in dimethylformamide (6 mL, 65 mmol) was added (3R)-1-[(tert-butoxy) carbonyl]piperidine-3-carboxylic acid (1.23 g, 5.35 mmol), tripropyl-1,3,5,2×5,42.5,62.5-trioxatriphosphinane-2,4,6-trione (3.5 mL, 8.0 mmol) and ethylbis(propan-2-yl)amine (4.6 mL, 26.8 mmol) sequentially at room temperature. The reaction mixture was stirred for 16 h. After completion of the reaction (monitored by TLC), the reaction was diluted with water (25 mL) and extracted with ethyl acetate (2×50 mL). The organic layer was dried over Na₂SO₄ and concentrated under reduced pressure to get crude. The crude material was purified by flash chromatography on silica gel to eluent (20% EtOAc in hexane) to obtain the desired compound tert-butyl (3R)-3-((4,6-difluorobenzo[d]thiazol-2-yl) carbamoyl) piperidine-1-carboxylate (0.9 g, 41% yield). ¹H-NMR (400 MHz; DMSO-d₆.): δ 12.75 (S, 1H), 7.81 (dd, J=2.0 Hz, 8.4 Hz, 1H), 7.38 (dd, J=2.4 Hz, 10.8 Hz, 1H), 3.6-3.9 (m, 2H), 2.85-2.95 (m, 2H), 2.6-2.7 (m, 1H), 1.9-2.0 (m, 1H), 1.6-1.8 (m, 3H), 1.45 (s, 9H)

Step-2: Synthesis of(S)-N-(4.6-difluorobenzo[d]thiazol-2-yl) piperidine-3-carboxamide hydrochloride: To an oven dry round bottom flask tert-butyl (3S)-3-[(4,6-difluoro-1,3-benzothiazol-2-yl) carbamoyl]piperidine-1-carboxylate (420 mg, 1.06 mmol) in DCM (5.0 mL) was added 4M HCl in dioxane (2 mL) at 0° C., then stirred at RT for 3 h. After completion, the reaction mixture was concentrated to get residue, then triturated with ether to get the desired compound (3S)—N-(4,6-difluorobenzo[d]thiazol-2-yl) piperidine-3-carboxamide hydrochloride (0.330 g, 94% yield). ¹H NMR (DMSO-d₆, 400 MHZ): δ 12.92 (s, 1H), 9.32 (bs, 2H), 7.74 (d, J=8.4 Hz, 1H), 7.28 (t, J=10.8, 1H), 3.35-3.45 (m, 1H), 3.0-3.25 (m, 3H), 2.8-2.9 (m, 1H), 2.05-2.15 (m, 1H), 1.7-1.9 (m, 2H), 1.55-1.65 (m, 1H)

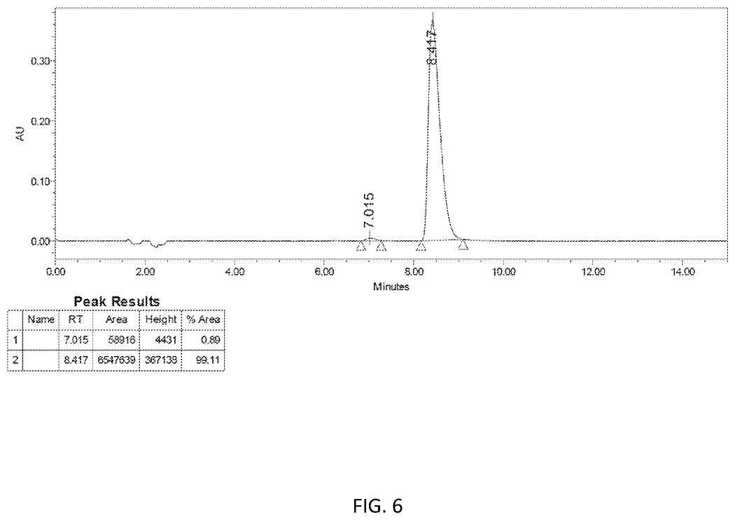

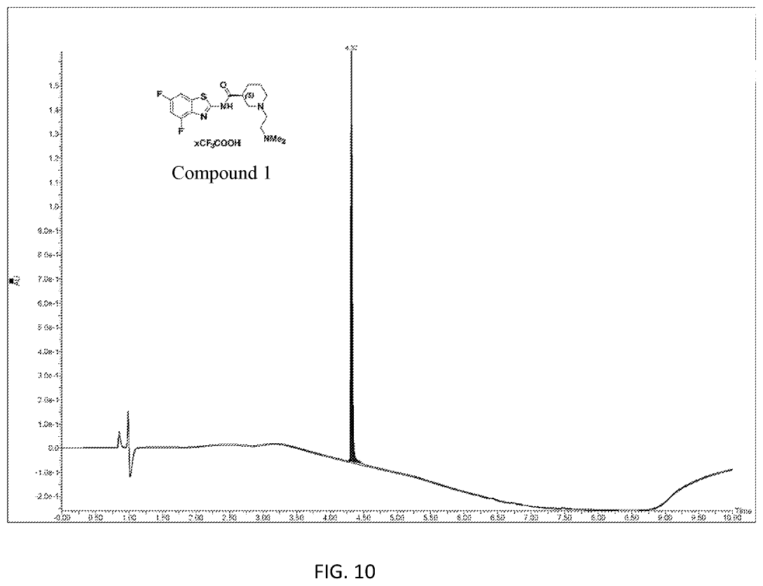

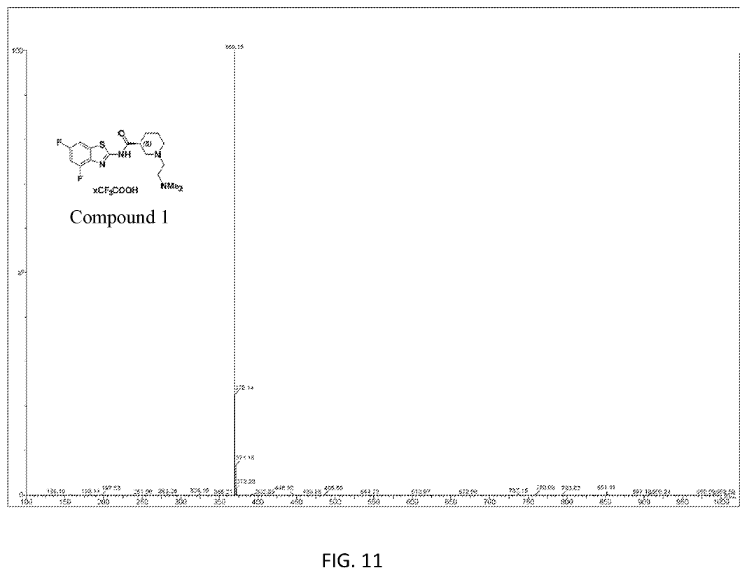

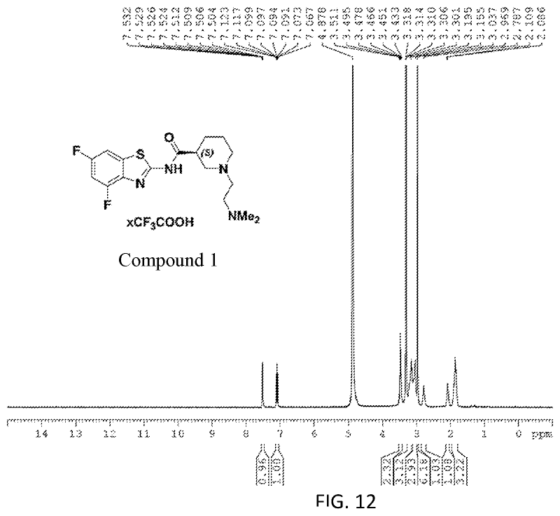

Step-3: Synthesis of(S)-N-(4.6-difluorobenzo[d]thiazol-2-yl)-1-(2-(dimethylamino)ethyl) piperidine-3-carboxamide: 2-chloro-N,N-dimethylethan-1-amine hydrochloride (515 mg, 3.60 mmol) was added to the stirred suspension of (3R)—N-(4,6-difluoro-1,3-benzothiazol-2-yl) piperidine-3-carboxamide hydrochloride (1 g, 3.0 mmol) and K₂CO₃ (1.65 g, 10.5 mmol) in acetonitrile (25 mL) at room temperature. The resulting mixture was stirred at 60° C. overnight and the progress of the reaction was monitored by TLC. The reaction mixture was concentrated under vacuum to get crude. The crude was purified by prep HPLC to obtain the desired compound TFA salt of(S)-N-(4,6-difluorobenzo[d]thiazol-2-yl)-1-(2-(dimethylamino)ethyl) piperidine-3-carboxamide (497 mg, 22.5%) as a white solid. LCMS Calculated: m/z 368.45. LCMS Observed: m/z 369.22 [M+H]⁺. LCMS chromatogram is provided in FIG. 10. MS result is provided in FIG. 11. ¹H NMR is provided in FIG. 12. Chiral LC chromatogram is provided in FIG. 6. ¹H NMR (MeOD, 400 MHZ): δ 7.50-7.54 (m, 1H), 7.0-7.15 (m, 1H), 3.45-3.55 (m, 2H), 2.9-3.3 (m, 12H), 2.75-2.90 (m, 1H), 2.05-2.20 (m, 1H), 1.8-1.96 (m, 3H). Prep purification method: Column: 1. Column: Atlantis T3 (19×250), 10 μm. Mobile phase: A, 0.1% TFA in water; B, ACN. Flow mode: 18 ml/min. Sample preparation diluents: ACN, H₂O. Wavelength: 214 nm.

### Example 5—Preparation of Trifluoroacetate Salt of Compound 2

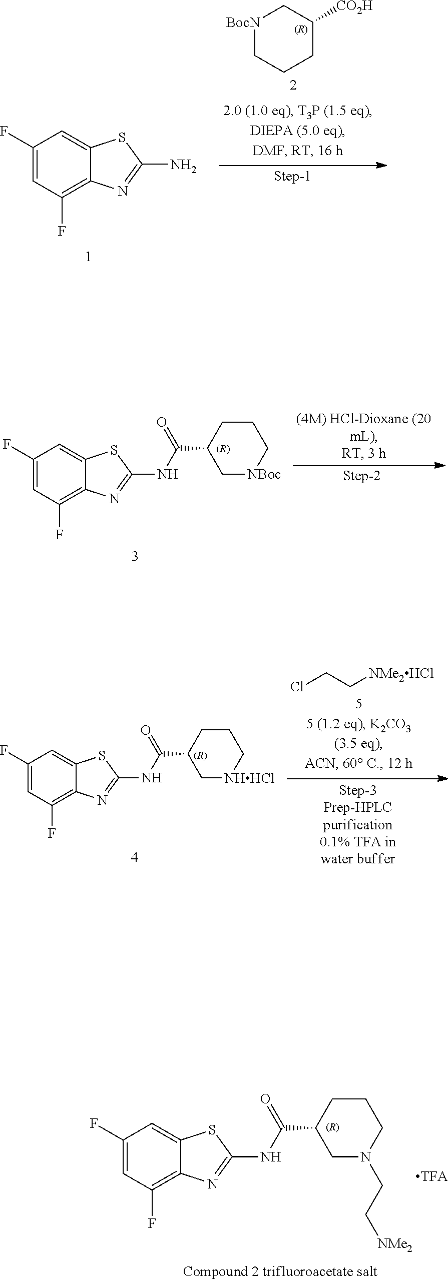

Step-1: Synthesis of tert-butyl (3R)-3-((4.6-difluorobenzo[d]thiazol-2-yl) carbamoyl) piperidine-1-carboxylate: To a stirred solution of 4,6-difluoro-1,3-benzothiazol-2-amine (2 g, 10.7 mmol) in dimethylformamide (10 mL, 129 mmol) was added (3R)-1-[(tert-butoxy) carbonyl]piperidine-3-carboxylic acid (2.46 g, 10.7 mmol), tripropyl-1,3,5,27.3,47.5,62.5-trioxatriphosphinane-2,4,6-trione (6.93 mL, 16.1 mmol) and ethylbis(propan-2-yl)amine (9.38 mL, 53.7 mmol) sequentially at room temperature. The reaction mixture was stirred for 16 h. After completion of the reaction (monitored by TLC), the reaction was diluted with water (50) mL) and extracted with ethyl acetate (2×100 mL). The organic layer was dried over Na₂SO₄ and concentrated under reduced pressure to get crude. The crude material was purified by flash chromatography on silica gel to eluent (20% EtOAc in hexane) to obtain the desired compound tert-butyl (3R)-3-((4,6-difluorobenzo[d]thiazol-2-yl) carbamoyl) piperidine-1-carboxylate (1.8 g. 42% yield). ¹H-NMR (400 MHZ: DMSO-d₆.): δ 12.75 (s, 1H), 7.81 (dd, J=2.0 Hz, 8.4 Hz, 1H), 7.38 (dd, J=2.4 Hz, 10.8 Hz, 1H), 3.6-3.9 (m, 2H), 2.85-2.95 (m, 2H), 2.6-2.7 (m, 1H), 1.9-2.0 (m, 1H), 1.6-1.8 (m, 3H), 1.45 (s, 9H)

Step-2: Synthesis of (3R)—N-(4.6-difluorobenzo/d]thiazol-2-yl) piperidine-3-carboxamide hydrochloride: To an oven dry round bottom flask tert-butyl (3R)-3-[(4,6-difluoro-1,3-benzothiazol-2-yl) carbamoyl]piperidine-1-carboxylate (440 mg, 1.11 mmol) in DCM (5.0 mL) was added 4M HCl in dioxane (2 mL) at 0)° C., then stirred at RT for 6 h. After completion, the reaction mixture was concentrated to get residue, then triturated with ether to get desired compound (3R)—N-(4,6-difluorobenzo[d]thiazol-2-yl) piperidine-3-carboxamide hydrochloride (310 mg, 88% yield). ¹H NMR (DMSO-d₆, 400 MHZ) δ:12.89 (bs, 1H), 8.98 (bs, 2H), 7.82 (d, J=8.4 Hz, 1H), 7.40 (t. J=10.8, 1H), 3.25-3.35 (m, 1H), 3.2-3.1 (m, 1H), 2.95-2.7 (m, 3H), 2.05-1.95 (m, 1H), 1.9-1.62 (m, 2H), 1.6-1.5 (m, 1H).

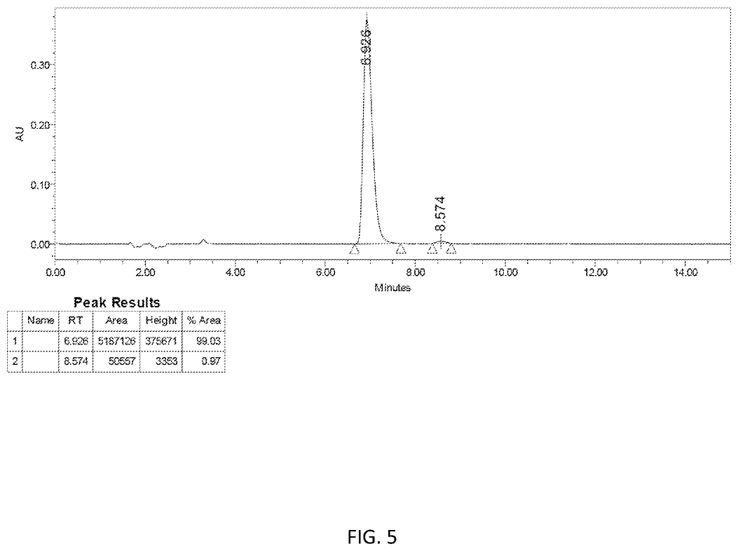

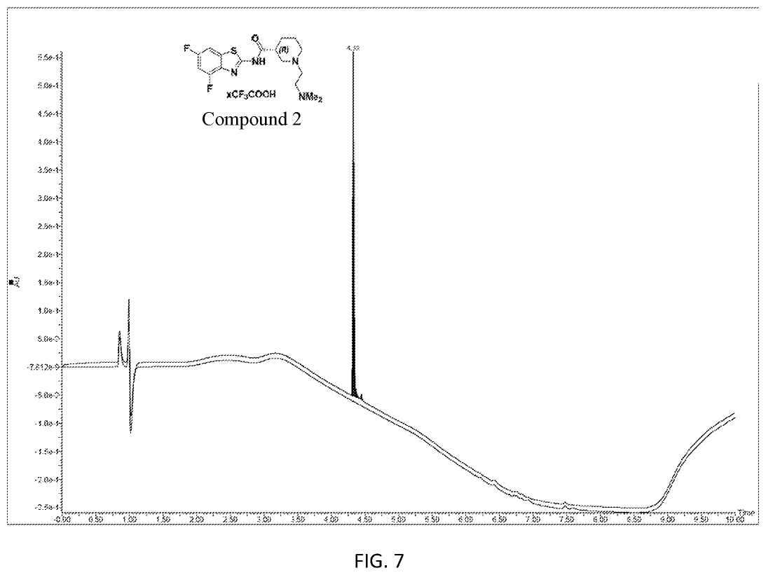

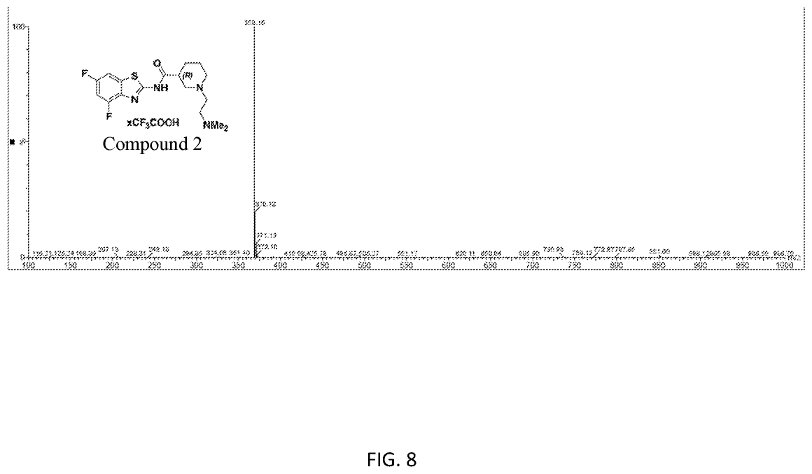

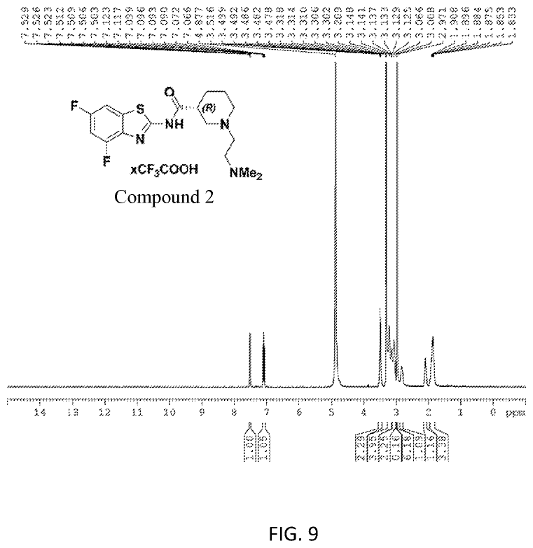

Step-3: Synthesis of (R)-N-(4.6-difluorobenzo[d]thiazol-2-yl)-1-(2-(dimethylamino)ethyl) piperidine-3-carboxamide: 2-Chloro-N,N-dimethylethan-1-amine hydrochloride (257 mg, 1.8 mmol) was added to the stirred suspension of (3R)-N-(4,6-difluoro-1,3-benzothiazol-2-yl) piperidine-3-carboxamide hydrochloride (0.5 g. 1.5 mmol) and K₂CO₃ (0.825 g. 5.23 mmol) in acetonitrile (12 mL) at room temperature. The resulting mixture was stirred at 60° C. overnight and the progress of the reaction was monitored by TLC. The reaction mixture was concentrated under vacuum to get crude. The crude was purified by prep HPLC using 0.1% TFA in water buffer to obtain the desired compound as TFA salt of (R)-N-(4,6-difluorobenzo[d]thiazol-2-yl)-1-(2-(dimethylamino)ethyl) piperidine-3-carboxamide (240) mg. 21.5%) as a white solid. LCMS Calculated: m/z. 368.45. LCMS Observed: m/z 369.22 [M+H]*. LCMS chromatogram is provided in FIG. 7. MS result is provided in FIG. 8. ¹H NMR is provided in FIG. 9. Chiral LC chromatogram is provided in FIG. 5. ¹H NMR (CD₃OD, 400 MHZ): δ 7.50-7.53 (m, 1H), 7.06-7.13 (m, 1H), 3.2-3.3 (m, 2H), 2.85-3.10 (m, 12H), 2.60-2.73 (m, 1H), 2.10-1.98 (m, 1H), 1.70-1.96 (m, 3H) ppm. Prep purification method: Column: 1. Column: Atlantis T3 (19×250), 10 μm. Mobile phase: A, 0.1% TFA in water: B, ACN. Flow mode: 18 ml/min. Sample preparation diluents: ACN, H₂O. Wavelength: 214 nm.

### Example 6—Preparation of Acetate Salt of Compound 1

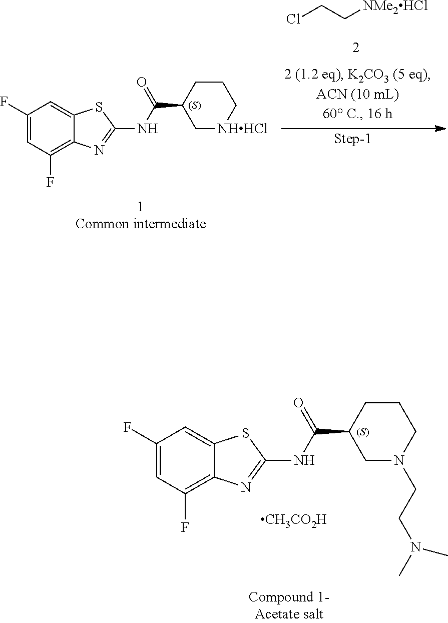

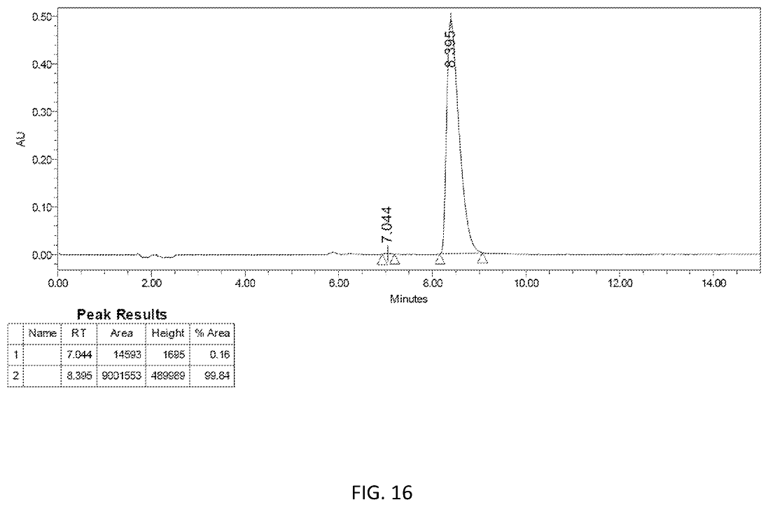

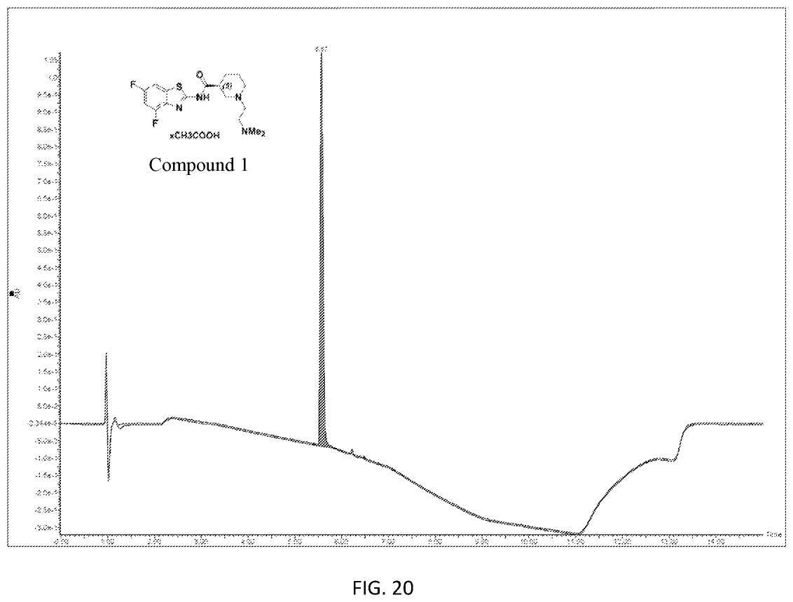

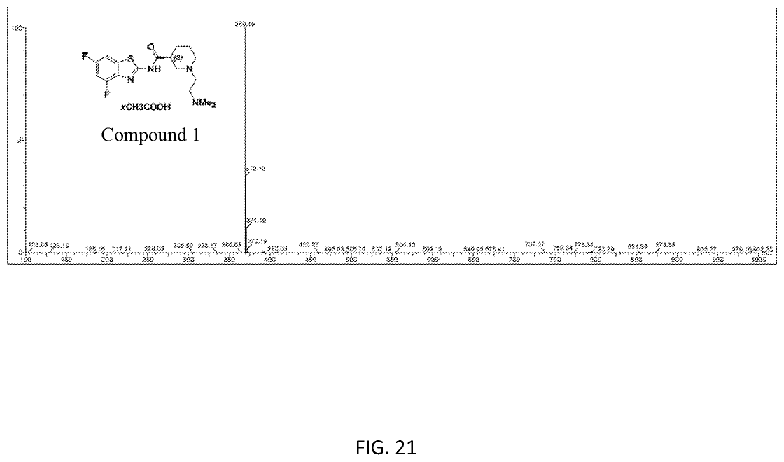

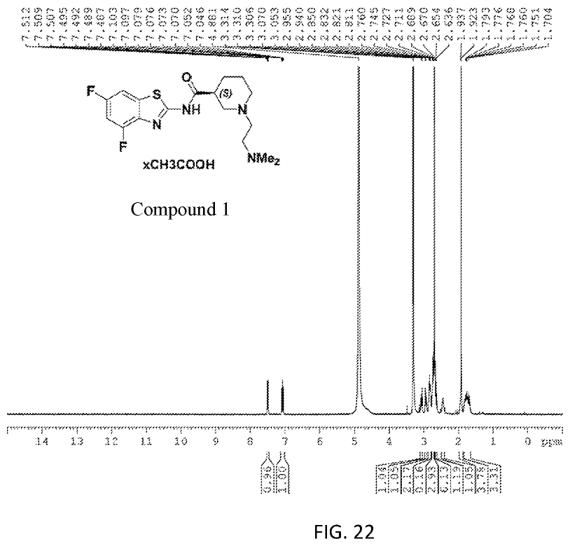

Step-1: Synthesis of(S)-N-(4.6-difluorobenzo[d]thiazol-2-yl)-1-(2-(dimethylamino)ethyl) piperidine-3-carboxamide: 2-Chloro-N,N-dimethylethan-1-amine hydrochloride (407 mg, 2.83 mmol) was added to the stirred suspension of(S)-N-(4,6-difluoro-1,3-benzothiazol-2-yl) piperidine-3-carboxamide (prepared as in Example 4 above) (0.7 g, 2.35 mmol) and K₂CO₃ (1.63 g, 11.8 mmol) in acetonitrile (10 mL) at room temperature. The resulting mixture was stirred at 60° C. for 16 h. The progress of the reaction was monitored by TLC. The reaction mixture was concentrated under vacuum to get crude. The crude was purified by prep-HPLC using 5 mMol ammonium acetate in water as buffer to obtain the desired compound acetate salt of(S)-N-(4,6-difluoro-1,3-benzothiazol-2-yl)-1-[2-(dimethylamino)ethyl]piperidine-3-carboxamide (150 mg, 17% yield) as a white solid. LCMS Calculated: m/z 368.45. LCMS Observed: m/z 369.15 [M+H]⁺. LCMS chromatogram is provided in FIG. 20. MS result is provided in FIG. 21. ¹H NMR is provided in FIG. 22. Chiral LC chromatogram is provided in FIG. 16. ¹H NMR (CD₃OD, 400 MHZ) 8:7.49-7.51 (m, 1H), 7.04-7.11 (m, 1H), 3.05-3.15 (m, 1H), 2.9-3.0 (m, 1H), 2.8-2.9 (m, 2H), 2.8-2.6 (m, 4H), 2.4-2.5 (m, 1H), 1.93 (s, 6H), 1.70-1.63 (m, 4H). Prep purification method: Column: X BRIDGE SHIELD (19×250) mm, 10 μm. Mobile phase: A, 5 mM Ammonium acetate in water; B, ACN. Flow mode: 18 ml/min. Sample preparation diluents: ACN, H₂O. Wavelength: 214 nm.

### Example 7—Preparation of Acetate Salt of Compound 2

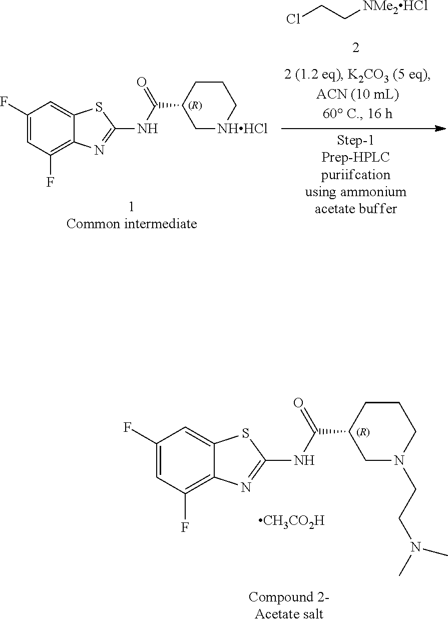

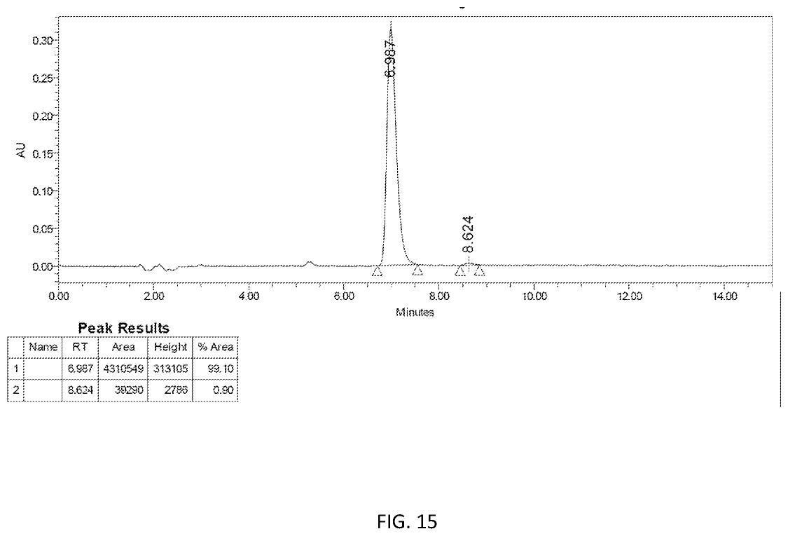

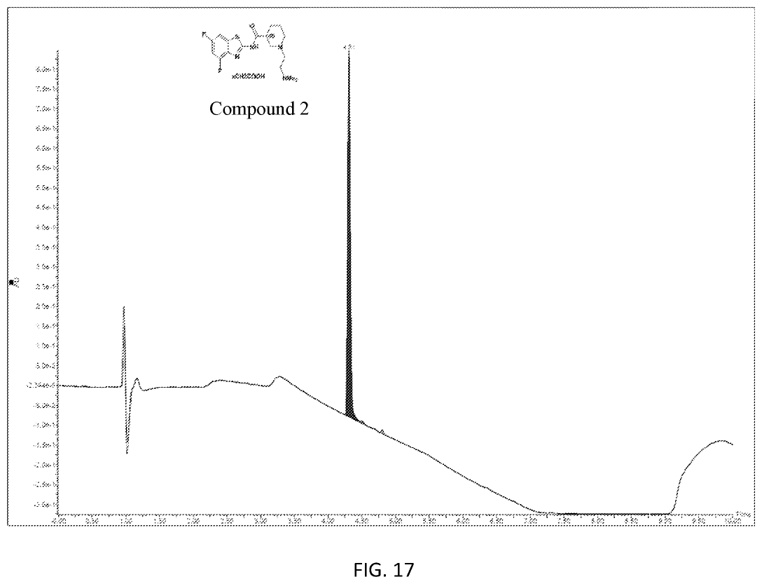

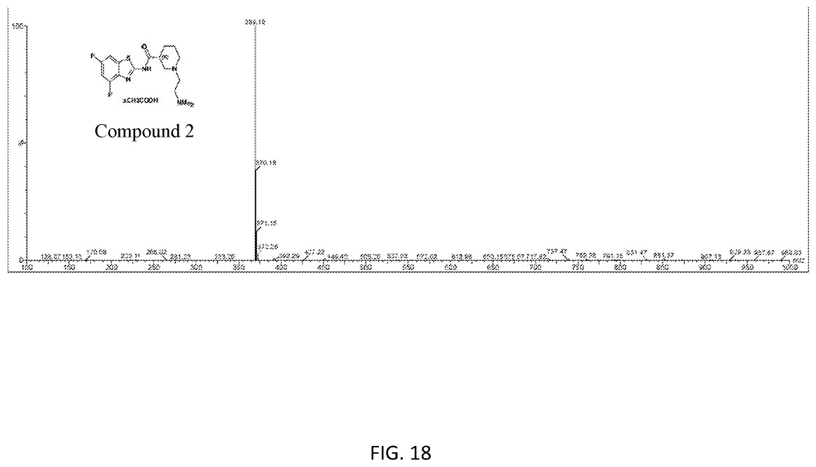

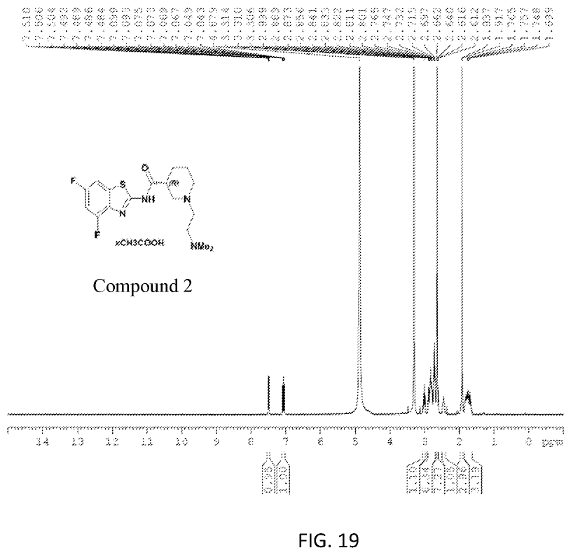

Step-1: Synthesis of (R)-N-(4.6-difluorobenzo[d]thiazol-2-yl)-1-(2-(dimethylamino)ethyl) piperidine-3-carboxamide: 2-Chloro-N,N-dimethylethan-1-amine hydrochloride (203 mg, 1.41 mmol) was added to the stirred suspension of (R)-N-(4,6-difluoro-1,3-benzothiazol-2-yl) piperidine-3-carboxamide (prepared as in Example 5 above) (0.35 g. 1.17 mmol) and K₂CO₃ (0.81 g. 5.9 mmol) in acetonitrile (6 mL) at room temperature. The resulting mixture was stirred at 60° C. for 16 h. The progress of the reaction was monitored by TLC and LCMS. The reaction mixture was concentrated under vacuum to get crude. The crude was purified by prep-HPLC by using 5 mMol ammonium acetate buffer in water to obtain the desired compound as a acetate salt of (R)-N-(4,6-difluoro-1,3-benzothiazol-2-yl)-1-[2-(dimethylamino)ethyl]piperidine-3-carboxamide (75 mg, 17% yield) as a white solid. LCMS Calculated: m/z. 368.45. LCMS Observed: m/z 369.15 [M+H]⁺. LCMS chromatogram is provided in FIG. 17. MS result is provided in FIG. 18. ¹H NMR is provided in FIG. 19. Chiral LC chromatogram is provided in FIG. 15. ¹H NMR (CD₃OD, 400 MHZ) δ:7.48-7.51 (m, 1H), 7.04-7.1 (m, 1H), 3.0-2.9 (m, 1H), 2.9-2.6 (m, 7H), 2.4-2.5 (m, 1H), 1.92 (m, 6H), 1.9-1.45 (m, 4H). Prep purification method: Column: Atlantis T3 (19×250), 10 μm. Mobile phase: A, 0.1% TFA in water: B, ACN. Flow mode: 18 ml/min. Sample preparation diluents: ACN, H₂O. Wavelength: 214 nm.

### Example 8—Materials and Methods

EC₅₀ and Eₘₐₓ CGMP Determination

Test compounds were evaluated for potentiation of cGMP in human GC-A overexpressing HEK293 cells (Cardiorenal Research Laboratory, Mayo Clinic). Test compounds at 10 mM DMSO stock concentration were dispensed into 384 well small volume white plates using Tecan D300e digital dispenser starting at 5 μM or 40 μM concentration and diluted 2-fold for 10-point concentration response. Wells were backfilled with DMSO such that the final concentration of DMSO in all wells was maintained at 0.3% DMSO. Atrial natriuretic peptide (ANP, Phoenix Pharmaceuticals) ANP was prepared as working stock aliquots at 5 μM in phosphate-buffered solution (PBS) with 0.1% bovine serum albumin (BSA) and diluted to 0.1 mM in buffer plus Tween20 at 0.3% and added to plates to a final concentration equivalent to EC₂₀ ANP in the cGMP assay. GC-A cells in suspension (10 ml) in OptiMem media (ThermoFisher 11058-021) supplemented with 2% fetal bovine serum were plated at 1×10⁶ cells/mL and assay plates were spun at 1000 rpm for 30 sec and incubated at room temperature for 30 min. GC-A-mediated activation of cGMP production was measured using cGMP CisBio detection homogenous time-resolved florescence (HTRF) competition assay using labeled-cGMP in human GC-A-overexpressing HEK293 cells. 5 μL of d-2 acceptor was dispensed into each well followed by addition of 5 μL of Anti-cGMP Eu-Cryptate antibody donor and plates were spun at 1000 rpm for 30 sec and incubated in the dark at room temperature for 1 hr. HTRF florescence was read on the ClarioStar microplate reader (BMG). A 100% response was determined from wells in the absence of compound and presence of saturating concentration of ANP (10 nM), and 0) % response was determined from wells containing an EC₂₀ concentration of ANP determined for each new lot of ANP. Concentration-response curves for test compounds were analyzed by linear regression analysis using GraphPad software to determine EC₅₀ and Eₘₐₓ values.

### GC-A Binding Studies for Compounds 1-3 Acetate Salt Forms

Surface plasmon resonance (SPR) measurements were performed at 25° C. on a BI-4500 SPR instrument (Biosensing Instrument Inc. Tempe AZ). As per the instructions by the Biosensing instrument manual, 400 mM nickel sulfate in deionized water was linked to the Ni-NTA sensor chip (Biosensing Instrument Inc. Tempe AZ. Then 30 μg/ml of extracellular domain human GC-A recombinant protein (My BioSource, Inc. San Diego, CA), containing 12 histidine residues on the C-terminus, was then immobilized to the nickel sulfate on the Ni-NTA sensor chip. After, the chip was washed with buffer (150 mM NaCl, 50 μM EDTA pH 7.4, 0).1% DMSO), then 100 μL of sequentially diluted Compound 3 acetate salt (0.125, 0.25, 0.5, 1, 2 μM), Compound 1 acetate salt (6.25, 12.5, 25, 50, 100 nM) or Compound 2 acetate salt (0.125, 0.25, 0.5, 1, 2 μM) was injected at the rate of 60 ul/min and allowed to dissociate for 60 seconds. Data was collected as sensorgrams. Binding kinetics were derived from sensorgrams using BI-Data Analysis Program (Biosensing Instrument, Tempe AZ). Affinity analysis of GC-A with Compound 3, Compound 2 and/or Compound 1 interactions were performed using a 1:1 Langmuir binding model. Two series were performed for all studies.

### CGMP Levels in HEK293 Cells Overexpressing Human GC-A

HEK293 GC-A cells were seeded in 48-well plates (1×10⁵ cell per well) and cultured overnight to reach 80-90% confluency. The treatment buffer that consists of Hank's Balanced Salt Solution (HBSS), 0.1% BSA, 2 mM HEPES and 0.5 mM 3-isobutyl-1-methylzanthine (IBMX—a non-specific phosphodiesterase inhibitor), was used in all experiments. Compound 3, Compound 1, and Compound 2 acetate salt were dissolved in DMSO and ANP was dissolved in phosphate buffered solution (PBS). On the experimental day, growth media was replaced with treatment buffer and HEK293 GC-A cells were 1) treated with Compound 3, Compound 1 or Compound 2 at doses of 1 or 10 μM (reaching a final concentration of 0).1% DMSO) alone for 5 min at 37° C.: or 2) pretreated with Compound 3, Compound 1 or Compound 2 at doses of 1 or 10 μM (reaching a final concentration of 0.1% DMSO) for 5 min at 37° C., followed by a treatment with ANP (10-10 M) for additional 10 min at 37° C. HEK293 GC-A cells were also treated with vehicle (0.1% DMSO) which served as a negative control. Then all cells were washed with PBS once and lysed with 0.1M HCl. Intracellular cGMP was measured in the lysate using a commercial cGMP ELISA kit (Enzo Life Sciences, Farmingdale, NY) as instructed by the manufacturer.

### CGMP Levels in Human Cardiomyocytes

Primary Human cardiomyocytes (HCMs: Catalog No. 6200, Lot No. 6288, PromoCell) were maintained and subcultured according to the manufacturer's protocols. Passage 5 was used in the study. Treatment buffer, Compound 3, Compound 1 and Compound 2 acetate salt, ANP, and vehicle were prepared as described above. Briefly, 5×10⁵ of cells/well were grown in 6-well plates until 80 to 90% confluency and were then pretreated with vehicle or Compound 3, Compound 1 or Compound 2 at doses of 1 or 10 μM for 5 min at 37° C., then cells were treated with ANP (10-10 M) for an additional 10 min at 37° C. Afterward, all cells were washed with PBS once and lysed with 0.1 M HCl at room temperature, and intracellular cGMP levels were measured in the lysate using a commercial cGMP ELISA kit (Enzo Life Sciences) with the acetylation method as instructed by the manufacturer.

### In Vivo Actions in Spontaneously Hypertensive Rats

The cGMP generating, blood pressure lowering and renal enhancing actions of a single intravenous (IV) bolus dose of Compound 3, Compound 1 or Compound 2 acetate salt (n=4/small molecule form: 10 mg/kg) or Vehicle (n=5: 15% DMSO/60% PEG300/25% PBS) in spontaneously hypertensive rats (SHRs; male: 12 weeks old) were investigated. Studies were performed in accordance with the Animal Welfare Act and with approval of the Mayo Clinic Institutional Animal Care and Use Committee.

SHRs were administered Inactin (100 mg/kg: IP) to induce anesthesia and then maintained with additional Inactin (100 mg/kg: IP), as required. Once adequate anesthesia was achieved, the anesthetized SHRs were placed on a heating table set at 37° C. to maintain body temperature for the entire study and then were subjected to vessel and bladder cannulation. A polyethylene (PE)-50 tube catheter was placed into the jugular vein for saline, Compound 3, Compound 1, Compound 2 or vehicle IV administration. The carotid artery was cannulated with a PE-50 tube catheter for blood pressure monitoring (Sonometrics, London, Ontario, Canada) and blood sampling/collection. The bladder was accessed and cannulated with a PE-90 tube catheter for passive urine collection. After completion of the procedural set up, a 45-minute equilibration period was performed that included continuous IV saline infusion, a 30-minute baseline (pre-bolus) urine collection and a single baseline blood sample at the end of the 30-minute baseline urine collection. Five minutes after the baseline blood sample, a single IV bolus of Compound 3, Compound 1 or Compound 2 (10 mg/kg) or vehicle was administered, followed by a 60-minute clearance (post-bolus) with continuous IV saline administration to collect urine and blood samples that included a 15-minute post-IV bolus blood sample. BP was monitored and recorded, beginning prior to IV bolus administration (0-minute post-bolus) and then at 15, 30, 45 and 60 minutes post-bolus. At the end of the 60-minute clearance, the anesthetized rats were euthanized by exsanguination and all blood and urine samples were stored at −80° C. until assayed. Plasma and urinary cGMP levels were measured using a cGMP ELISA kit (Enzo Life Sciences, Farmingdale, NY) as instructed by the manufacturer. Urinary sodium concentrations were determined with an electrolyte analyzer (Diamond Diagnostics Inc., Holliston, MA).

### Example 9—cGMP Generation in HEK 293 Cells Overexpressing Human GC-A Receptor

Measurements were performed as described in Example 8. Table 1 reports the cGMP generating abilities of Compound 3 TFA salt, Compound 3 acetate salt, Compound 2 TFA salt, Compound 1 TFA salt, Compound 2 acetate salt and Compound 1 acetate salt forms. Compound 3 acetate salt was more potent in potentiating ANP-mediated cGMP generation in HEK293 GC-A cells than Compound 3 TFA salt. Compound 1 was more potent in potentiating ANP-mediated cGMP generation in HEK293 GC-A cells than Compound 2.

TABLE 1
cGMP activities

| Salt Form | EC₅₀ in μM | E_{Max} (%) |
|---|---|---|
| Compound 3 TFA salt | 0.57 | 95.8 |
| Compound 3 acetate salt | 0.32 | 84.0 |
| Compound 2 TFA salt | 0.73 | 82.7 |
| Compound 1 TFA salt | 0.16 | 75.3 |
| Compound 2 acetate salt | 1.32 | 73.1 |
| Compound 1 acetate salt | 0.15 | 81.5 |

### Example 10—Surface Plasmon Resonance Binding of Compounds 1-3 Acetate Salt Forms to the GC-A Receptor

Measurements were performed as described in Example 8. As shown in Table 2, Compound 1 acetate salt showed greater binding for the GC-A receptor than Compound 3 acetate salt and Compound 2 acetate salt. The K_{D} value for Compound 1 acetate salt was 12 nM, while K_{D} value for Compound 3 acetate salt and Compound 2 acetate salt were 176 nM and 120 nM, respectively. These data demonstrate that Compound 1 acetate salt has superior binding to human GCA than Compound 3 acetate salt or Compound 2 acetate salt.

TABLE 2
Surface Plasmon Resonance Binding Kinetics for Compounds 1-3 Acetate
Salt to Human GC-A Receptor

| | kₐ (M⁻¹s⁻¹) | k_{d} (s⁻¹) | K_{D} (nM) |
|---|---|---|---|
| Compound 3 | 1.13 × 10⁵ | 2.0 × 10⁻² | 176 |
| Compound 1 | 1.09 × 10⁶ | 1.4 × 10⁻² | 12 |
| Compound 2 | 4.3 × 10⁵ | 5.2 × 10⁻² | 121 |

kₐ = association rate; k_{d} = dissociation rate; K_{D} = equilibrium dissociation rate.

### Example 11—cGMP Generation in HEK 293 Cells Overexpressing Human GC-A Receptor and Human Cardiomyocytes with Compound 3 Acetate Salt

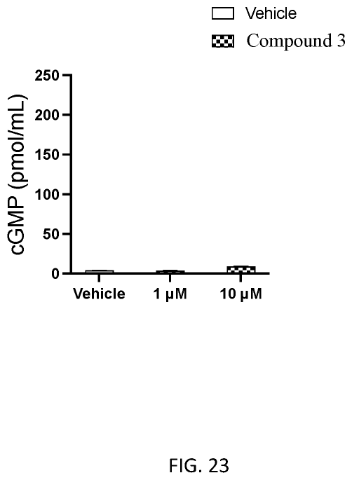

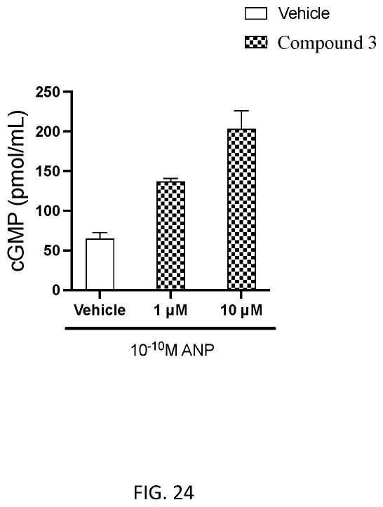

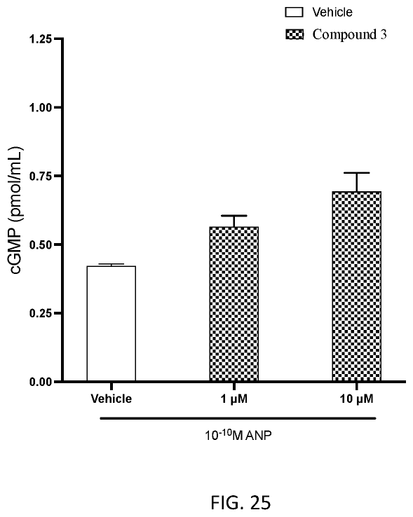

Measurements were performed as described in Example 8. FIG. 23 shows cGMP generation to vehicle or Compound 3 (1 or 10 μM) treatment in HEK 293 cells overexpressing human GC-A. There was a no effect of on cGMP generation with Compound 3. FIG. 24 shows a dose dependent increase in cGMP generation with Compound 3, in the presence of ANP (10⁻¹⁰ M), in HEK 293 cells overexpressing human GC-A. This dose dependent increase in cGMP generation with Compound 3 acetate salt, in the presence of ANP (10-10 M), was also observed in HCMs (FIG. 25).

### Example 12—cGMP Generation, Blood Pressure and Renal Function in Spontaneously Hypertensive Rats with Compound 3 Acetate Salt

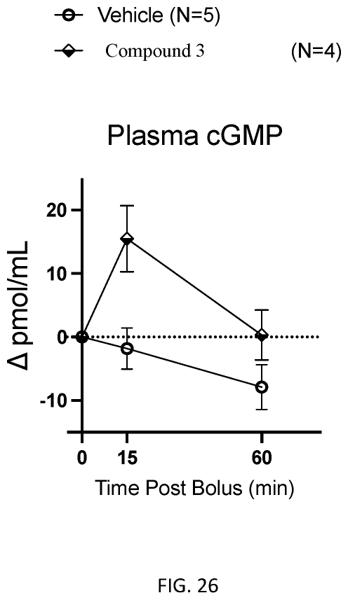

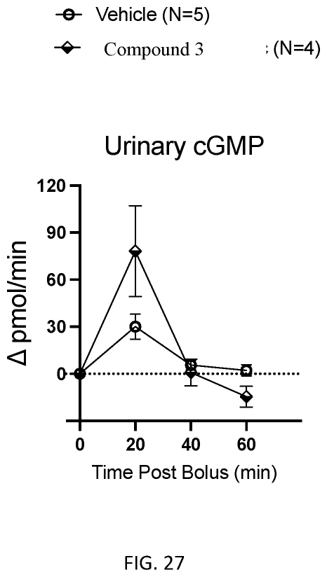

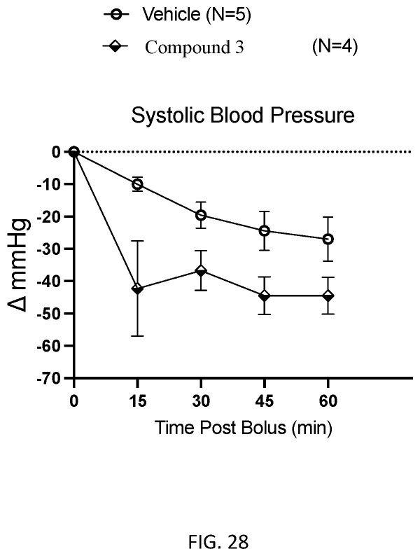

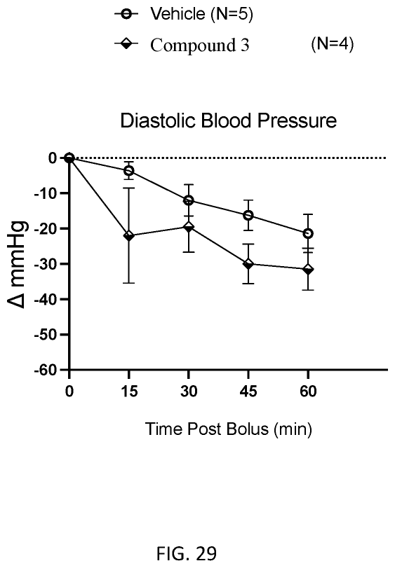

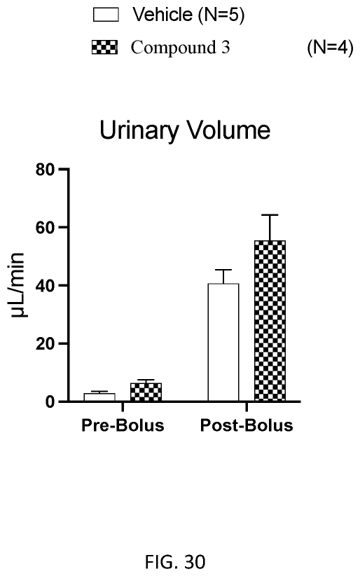

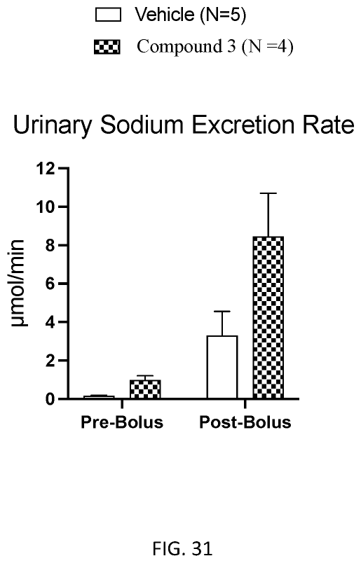

Measurements were performed as described in Example 8. A single IV bolus of Compound 3 at a dose of 10 mg/kg produced an increase in plasma (FIG. 26) and urinary (FIG. 27) cGMP at 15 minutes after administration, thus supporting GC-A target engagement in vivo. This increase in plasma and urinary cGMP with Compound 3 resulted in greater reduction in systolic (FIG. 28) and diastolic (FIG. 29) blood pressure as well as a greater increase in urinary volume (FIG. 30) and urinary sodium excretion (FIG. 31) compared to vehicle.

### Example 13—cGMP Generation in HEK 293 Cells Overexpressing Human GC-A Receptor with Compound 1 and Compound 2 Acetate Salt

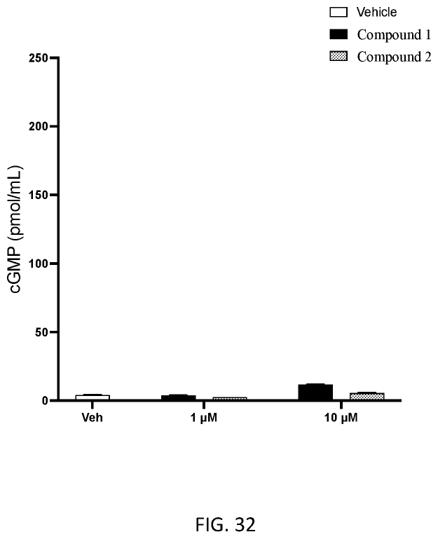

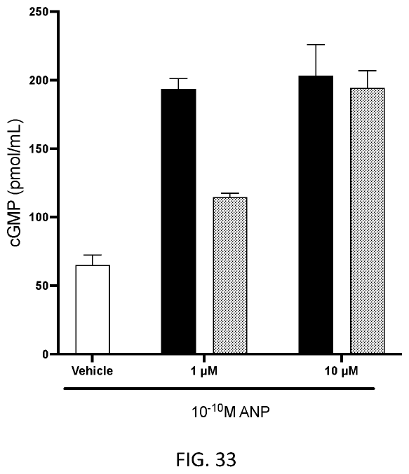

Measurements were performed as described in Example 8. FIG. 32 shows cGMP generation to vehicle. Compound 1 or Compound 2 treatment in HEK 293 cells overexpressing human GC-A. The cGMP generation with Compound 1 and Compound 2 was similar to vehicle. FIG. 33 shows cGMP generation to vehicle. Compound 1 or Compound 2 treatment, in the presence of ANP (10-10 M), in HEK 293 cells overexpressing human GC-A. There is a dose response increase in cGMP generation with Compound 1 and Compound 2. Notably, the cGMP generation is higher with Compound 1, than Compound 2, at both doses.

### Example 14—cGMP Generation in Primary Human Cardiomyocytes with Compound 1 and Compound 2 Acetate Salt

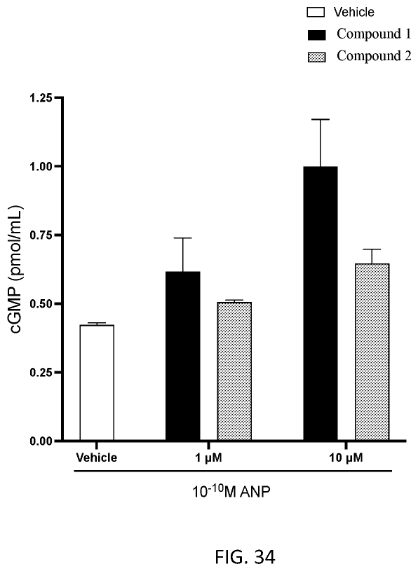

Measurements were performed as described in Example 8. FIG. 34 shows cGMP generation to vehicle or Compound 1 or Compound 2 in the presence of ANP (10-10 M) in HCMs. Both Compound 1 and Compound 2 increased cGMP levels in a dose dependent manner compared to vehicle, however Compound 1 was more potent in generating cGMP in HCMs than Compound 2 at both doses.

### Example 15—cGMP Generation in Spontaneously Hypertensive Rats with Compound 1 and Compound 2 Acetate Salt

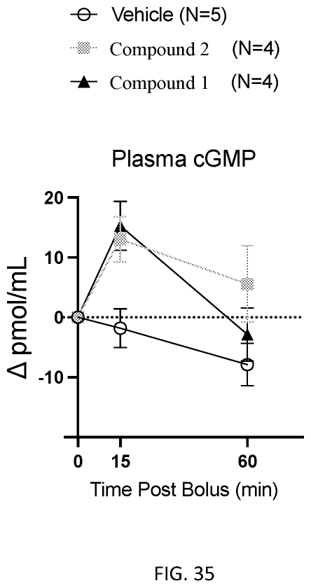

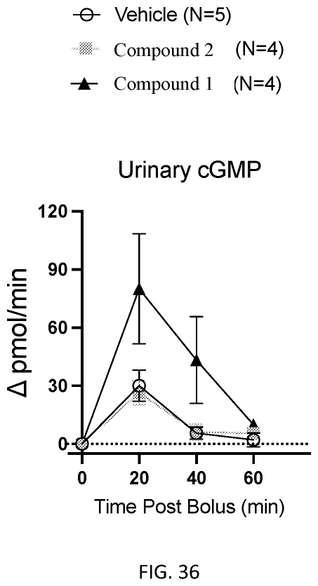

Measurements were performed as described in Example 8. A single IV bolus of Compound 2 or Compound 1 at a dose of 10 mg/kg produced an increase in plasma cGMP (FIG. 35) at 15 minutes after administration, thus supporting GC-A target engagement in vivo with both Compound 1 and Compound 2. Interestingly, a single IV bolus of Compound 1 at a dose of 10 mg/kg increased urinary cGMP (FIG. 36), however Compound 2 did not increase urinary cGMP (FIG. 36) and was similar to vehicle administration.

### Example 16—Reduction in Blood Pressure in Spontaneously Hypertensive Rats (SHRs) with Compound 1 and Compound 2 Acetate Salt

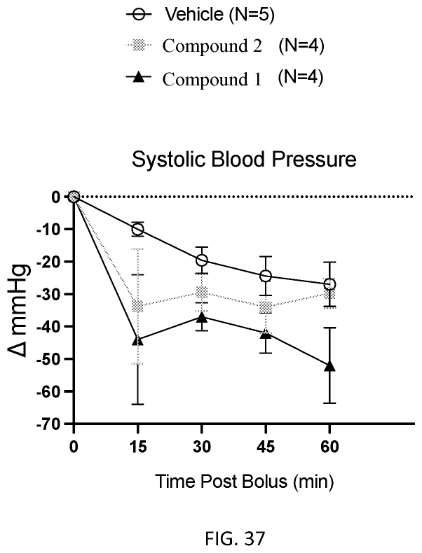

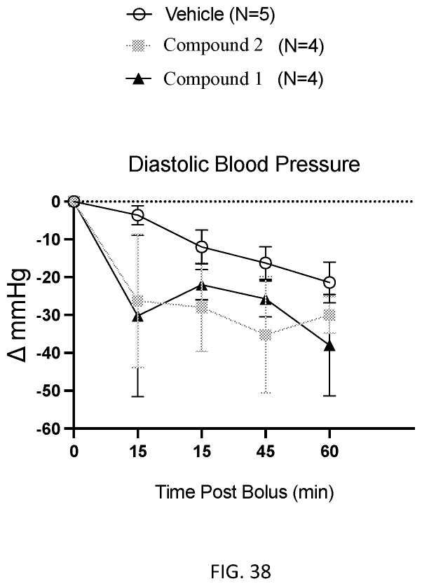

Measurements were performed as described in Example 8. A single IV bolus of Compound 1 at a dose of 10 mg/kg was a more effective anti-hypertensive compared to Compound 2 and vehicle. As shown in FIG. 37. Compound 1 was more effective at lowering systolic blood pressure (BP) than Compound 2 and vehicle, in SHRs throughout 60) minutes post bolus. As shown in FIG. 38. Compound 1 was more effective at lowering diastolic BP than Compound 2 and vehicle, in SHRs at 60 minutes post-bolus.

### Example 17—Renal Function in Spontaneously Hypertensive Rats with Compound 1 and Compound 2 Acetate Salt

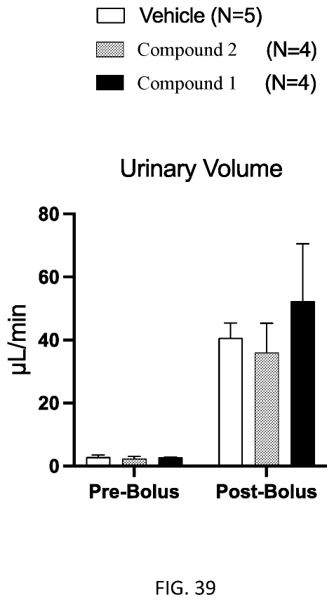

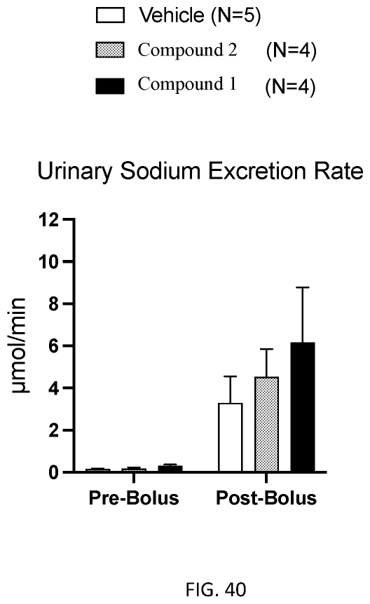

Measurements were performed as described in Example 8. A single IV bolus of Compound 1 at a dose of 10 mg/kg was more effective in enhancing renal function compared to Compound 2 and vehicle. As shown, Compound 1 was more effective in increasing urinary volume (FIG. 39) and urinary sodium excretion (FIG. 40), than Compound 2 and vehicle post-bolus.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound(S)-N-(4,6-difluorobenzo[d]thiazol-2-yl)-1-(2-(dimethylamino)ethyl) piperidine-3-carboxamide having formula: 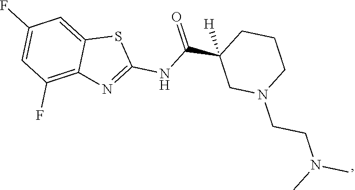 [SMILES: CN(C)CCN1CCC[C@H](C(=O)Nc2nc3c(F)cc(F)cc3s2)C1]
or a pharmaceutically acceptable salt thereof.

2. A compound (R)-N-(4,6-difluorobenzo[d]thiazol-2-yl)-1-(2-(dimethylamino)ethyl) piperidine-3-carboxamide having formula: 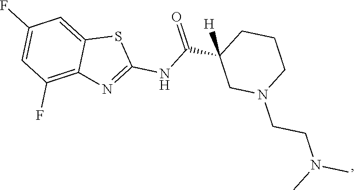 [SMILES: CN(C)CCN1CCC[C@@H](C(=O)Nc2nc3c(F)cc(F)cc3s2)C1]
or a pharmaceutically acceptable salt thereof.

3. A pharmaceutical composition comprising a compound or a pharmaceutically acceptable salt thereof, and a pharmaceutically acceptable carrier, wherein said compound is:
(a) (S)-N-(4,6-difluorobenzo[d]thiazol-2-yl)-1-(2-(dimethylamino)ethyl) piperidine-3-carboxamide having formula: 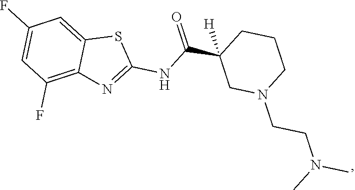 [SMILES: CN(C)CCN1CCC[C@H](C(=O)Nc2nc3c(F)cc(F)cc3s2)C1]
or
(b) (R)-N-(4,6-difluorobenzo[d]thiazol-2-yl)-1-(2-(dimethylamino)ethyl) piperidine-3-carboxamide having formula:

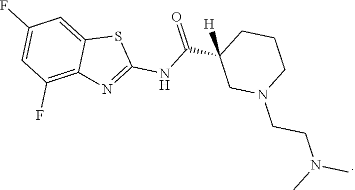 [SMILES: CN(C)CCN1CCC[C@@H](C(=O)Nc2nc3c(F)cc(F)cc3s2)C1]

4. A method of modulating particulate guanylyl cyclase receptor A (pGC-A) in a cell, the method comprising contacting the cell with an effective amount of a compound or a pharmaceutically acceptable salt thereof, wherein said compound is:
(a) (S)-N-(4,6-difluorobenzo[d]thiazol-2-yl)-1-(2-(dimethylamino)ethyl) piperidine-3-carboxamide having formula: 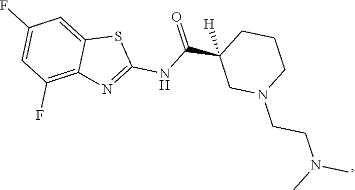 [SMILES: CN(C)CCN1CCC[C@H](C(=O)Nc2nc3c(F)cc(F)cc3s2)C1]
or
(b) (R)-N-(4.6-difluorobenzo[d]thiazol-2-yl)-1-(2-(dimethylamino)ethyl) piperidine-3-carboxamide having formula:

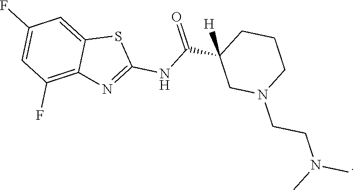 [SMILES: CN(C)CCN1CCC[C@@H](C(=O)Nc2nc3c(F)cc(F)cc3s2)C1]

5. The method of claim 4, comprising contacting the cell in vitro, in vivo, or ex vivo.

6. A method of modulating particulate guanylyl cyclase receptor A (pGC-A) in a subject, the method comprising administering to the subject in need thereof an effective amount of a compound or a pharmaceutically acceptable salt thereof, or the pharmaceutical composition of claim 3, wherein said compound is:
(a) (S)-N-(4,6-difluorobenzo[d]thiazol-2-yl)-1-(2-(dimethylamino)ethyl) piperidine-3-carboxamide having formula: 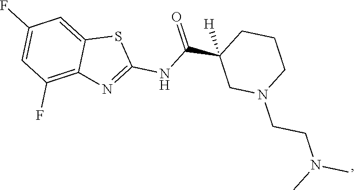 [SMILES: CN(C)CCN1CCC[C@H](C(=O)Nc2nc3c(F)cc(F)cc3s2)C1]
or
(b) (R)-N-(4,6-difluorobenzo[d]thiazol-2-yl)-1-(2-(dimethylamino)ethyl) piperidine-3-carboxamide having formula:

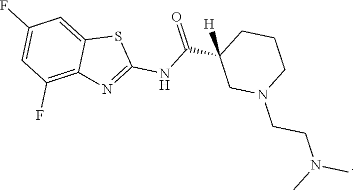 [SMILES: CN(C)CCN1CCC[C@@H](C(=O)Nc2nc3c(F)cc(F)cc3s2)C1]

7. The method of claim 6, wherein the modulating of the particulate guanylyl cyclase receptor A (pGC-A) comprises positive allosteric enhancement of activity of the particulate guanylyl cyclase receptor A (pGC-A).

8. A method of treating or preventing a disease or condition responsive to modulation of a particulate guanylyl cyclase receptor A (pGC-A) in a subject, the method comprising administering to the subject in need thereof a therapeutically effective amount of a compound or a pharmaceutically acceptable salt thereof, or the pharmaceutical composition of claim 3, wherein said compound is:
(a) (S)-N-(4,6-difluorobenzo[d]thiazol-2-yl)-1-(2-(dimethylamino)ethyl) piperidine-3-carboxamide having formula: 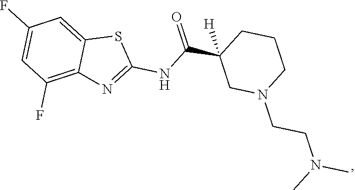 [SMILES: CN(C)CCN1CCC[C@H](C(=O)Nc2nc3c(F)cc(F)cc3s2)C1]
or
(b) (R)-N-(4,6-difluorobenzo[d]thiazol-2-yl)-1-(2-(dimethylamino)ethyl) piperidine-3-carboxamide having formula:

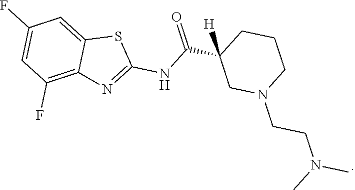 [SMILES: CN(C)CCN1CCC[C@@H](C(=O)Nc2nc3c(F)cc(F)cc3s2)C1]

9. The method of claim 8, wherein the disease or condition is a metabolic disease.

10. The method of claim 9, wherein the metabolic disease is selected from obesity, hypertriglyceridemia, metabolic syndrome, insulin resistance, hyperinsulinemia, diabetes, and acidemia.

11. The method of claim 8, wherein the disease or condition is a cardiovascular disease.

12. The method of claim 11, wherein the cardiovascular disease is selected from heart failure, cardiomyopathy, hypertension, high blood pressure, and myocardial infarction.

13. The method of claim 8, wherein the disease or condition is a kidney disease.

14. The method of claim 13, wherein the kidney disease is selected from nephropathy, acute renal failure, chronic kidney disease, and diabetic kidney disease.
